(19) United States
(12) Patent Application Publication
(10) Pub. No.: US 2006/0088894 A1
(43) Pub. Date: Apr. 27, 2006

(54) Prostate cancer biomarkers

(75) Inventors: George L Wright, Virginia Beach, VA (US); Bao-Ling Adam, Norfolk, VA (US); Yinsheng Qu, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Eastern Virginia Medical School, Norfolk, VA (US); Fred Hutchingson Cancer Research Center, Seattle, WA (US)

(21) Appl. No.: 10/513,649

(22) PCT Filed: May 9, 2003
(86) PCT No.: PCT/US03/14432

§ 371 Date: Sep. 1, 2005

Related U.S. Application Data
(60) Provisional application No. 60/379,018, filed on May 10, 2002.

(51) Int. Cl. G01N 33/574; G01N 33/00

(52) U.S. Cl. 435/7.23; 436/86

(57) ABSTRACT

Protein biomarkers that may advantageously be utilized in diagnosing prostate cancer, benign prostate hyperplasia or to make a negative diagnosis are described. Accordingly, in one aspect of the invention, methods for aiding in, or otherwise making, a diagnosis of prostate cancer or benign prostate hyperplasia are provided. In one form of the invention, a method includes detecting various protein biomarkers of defined molecular weight and correlating the detection to a diagnosis of prostate cancer, benign prostate hyperplasia or to a negative diagnosis. In yet another aspect of the invention, kits are provided that may be utilized to detect the biomarkers described herein. In a further of the invention, methods of using a plurality of classifiers to make a probable diagnosis of prostate cancer of benign prostate hyperplasia are provided. In certain forms of the invention, the methods include use of a boosted decision tree analysis. Various computer readable media are also provided.

### RELATED APPLICATION

This application claims priority from U.S. Provisional Application No. 60/379,018, filed May 10, 2002, which is incorporated herein by reference.

The present invention was made with Government support under grant number CA85067 awarded by the National Cancer Institutes Early Detection Research Network, grant number DAMD17-02-1-0054 awarded by the Department of Defense and a grant awarded by the Virginia Prostate Center.

### BACKGROUND OF THE INVENTION

The number of prostate cancer cases has tripled during the past decade due to the widespread use of serum prostate-specific antigen (PSA) testing and digital rectal examination [Howe et al., J. Natl. Cancer Inst. 93:824-842 (2001)]. Although these efforts have allowed for increased identification of individuals with cancer, overall “early” detection or determination of aggressive cancers is needed. PSA is currently the best over-all serum marker for prostate cancer (PCA) in clinical use. Nevertheless, the PSA test lacks specificity [Djavan, et al. Urology 54:517-5222, (1999); Pannek and Partin, Semin. Urol. Oncol. 16:100-105 (1998)], limiting its use as an early detection biomarker, and its relation to biologic activity has been questioned [Stamey et al., J. Urol. 167:103-111 (2002)]. It is important that additional diagnostic biomarkers be identified in order to reduce PCA mortality. However, because of the robust molecular and cellular heterogeneity of PCA, it is likely that a combination or a panel of biomarkers will be required to improve the early detection of prostate cancer.

The study of the cell's proteome presents a new horizon for biomarker discovery. Two-dimensional polyacrylamide gel electrophoresis (2D-EP) has been the classical approach to explore the proteome for separation and detection of differences in protein expression [Srinivas et al., Clin. Chem. 47:1901-1911 (2001); Adam et al., Proteomics 1:1264-1270 (2001)]. Advances in 2D-EP technology coupled with robotics and software programs for identifying potential protein alterations have improved this proteomic system. Nevertheless, 2D-EP is still cumbersome, labor intensive, suffers reproducibility problems, and is not readily transformed into a clinical assay. Advances have also been made in mass spectrometry to achieve high-throughput separation and analysis of proteins [Chong et al., Anal. Chem. 73:1219-1227 (2001); Ferrari et al., Rapid Comm. Mass Spec. 14:1149-1154 (2000); Keough et al. Electrophoresis 21:2252-2265 (2000)]. One of the recent advances is the ProteinChip® system manufactured by Ciphergen Biosystems, Inc. (Fremont, Calif.). This system uses surface-enhanced laser desorption/ionization time-of-flight (SELDI-TOF) mass spectrometry to detect proteins bound to a protein chip array [Merchant and Weinberger, Electrophoresis 21:1164-1177 (2000); Kuwata et al., Biochem. Biophys. Res. Comm. 245:764-773 (1998)].

This system is an extremely sensitive and rapid method to analyze complex mixtures of proteins and peptides. Initial studies from our laboratory established the potential of SELDI for discovery and profiling of prostate and bladder cancer biomarkers in body fluids and cell lysates [Wright et al., Prostate Cancer and Prostate Diseases 2:264-267 (1999); Vlahous et al., Am. J. Pathol., 158:1491-1502 (2001)]. However, further improved methods for differentiating between benign prostate hyperplasia, prostate cancer and a negative diagnosis are still needed. The present invention addresses this need.

### SUMMARY OF THE INVENTION

Protein biomarkers have been discovered that may be used to diagnose, or aid in the diagnosis of, prostate cancer or benign prostate hyperplasia, or to otherwise make a negative diagnosis. Accordingly, methods for aiding in the, or otherwise making a, diagnosis of prostate cancer or benign prostate hyperplasia are provided.

In one form of the invention, a method for aiding in the, or otherwise making a, diagnosis includes detecting at least two protein biomarkers in a test sample from a subject. The protein biomarkers have a molecular weight selected from the group consisting of about 4475±81, about 5074±91, about 5382±97, 7024±13, about 7820±14, about 8141±15, about 9149±16, about 9508±17, and about 9656±17 Daltons. The method further includes correlating the detection with a probable diagnosis of benign prostate hyperplasia, prostate cancer or a negative diagnosis.

In certain forms of the invention, the markers in a test sample from a subject may be detected in the following groups and may have the following molecular weights:

(i) about 7024±13 Dalton and about 7820±14 Dalton; (ii) about 7820±14 Dalton, about 7024±13 Dalton, about 5382±97 Dalton and about 4475±81 Dalton; (iii) about 8141±15 Dalton, about 9149±16 Dalton, and about 9656±17 Dalton; (iv) about 9149±16 Dalton and about 9508±17 Dalton; (v) about 5074±91 Dalton, about 9149±16 Dalton and about 9656±17 Dalton; or (vi) about 5382±97 Dalton, about 7024±13 Dalton and about 7820±14 Dalton. A correlation of the detection with a diagnosis of benign prostate hyperplasia, prostate cancer or a negative diagnosis can then be made by analyzing one or more of the above groups of protein biomarkers.

In yet another form of the invention, protein biomarkers that may be detected have molecular weights selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons.

At least two of the protein biomarkers described herein are typically detected. It is realized and described herein that one or more of the biomarkers may be detected and subsequently analyzed, including all of the biomarkers.

In yet another aspect of the invention, kits that may be utilized to detect the biomarkers described herein and may otherwise be used to diagnose, or otherwise aid in the diagnosis of, prostate cancer or benign prostate hyperplasia are provided. In one form of the invention, a kit may include a substrate comprising an adsorbent attached thereto, wherein the adsorbent is capable of retaining at least one protein biomarker selected from the group consisting of about 4475±81, about 5074±91, about 5382±97, 7024±13, about 7820±14, about 8141±15, about 9149±16, about 9508±17, and about 9656±17 Daltons; and instructions to detect the protein biomarker by contacting a test sample with the adsorbent and detecting the biomarker retained by the adsorbent.

In yet another embodiment of the invention, the kit may include a substrate comprising an adsorbent attached thereto, wherein the adsorbent is capable of retaining at least one protein biomarker selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons; and instructions to detect the protein biomarker by contacting a test sample with the adsorbent and detecting the biomarker retained by the adsorbent.

In yet another aspect of the invention, methods of using a plurality of classifiers to make a probable diagnosis of prostate cancer, benign prostate hyperplasia or a negative diagnosis are provided. In one form of the invention, a method includes a) obtaining mass spectra from a plurality of samples from normal subjects, subjects diagnosed with prostate cancer and subjects diagnosed with benign prostate hyperplasia; b) applying a boosted decision tree analysis to at least a portion of the mass spectra to obtain a plurality of weighted base classifiers comprising a peak intensity value and an associated threshold value; and c) making a probable diagnosis of at least one of prostate cancer, benign prostate hyperplasia and a negative diagnosis based on a linear combination of the plurality of weighted base classifiers. In certain forms of the invention, the method includes using the peak intensity value and the associated threshold value in linear combination to make a probable diagnosis of prostate cancer, benign prostate hyperplasia or to make a negative diagnosis.

In yet another aspect of the invention, computer program media storing computer instructions therein for instructing a computer to perform a computer-implemented process using a plurality of classifiers to make a probable diagnosis of prostate cancer, benign prostate hyperplasia, or a negative diagnosis, are provided. In one form of the invention, a computer program medium includes a) first computer program code means for obtaining mass spectra from a plurality of samples from normal subjects, subjects diagnosed with prostate cancer, and subjects diagnosed with benign prostate hyperplasia; b) second computer program code means for applying a boosted decision tree analysis to at least a portion of the mass spectra to obtain a plurality of weighted base classifiers comprising a peak intensity value and an associated threshold value; and c) third computer program code means for making a probable diagnosis of at least one of prostate cancer, benign prostate hyperplasia, and a negative diagnosis based on a linear combination of the plurality of weighted base classifiers. The peak intensity and associated threshold values may be used in linear combination to make a probable diagnosis of at least one of prostate cancer, benign prostate hyperplasia and a negative diagnosis.

In another form of the invention, a computer program medium includes (a) first computer program code means for detecting at least two protein biomarkers in a test sample from a subject, said protein biomarkers having a molecular weight selected from the group consisting of about 4475±81, about 5074±91, about 5382±97, 7024±13, about 7820±14, about 8141±15, about 9149±16, about 9508±17, and about 9656±17 Daltons; and (b) second computer program code means for correlating the detection with a probable diagnosis of benign prostate hyperplasia, prostate cancer or a negative diagnosis.

In a further form of the invention, a computer program medium includes a) first computer program code means for detecting in a test sample from a subject protein biomarkers in the following groups and having the following molecular weights: (i) about 7024±13 Dalton and about 7820±14 Dalton; (ii) about 7820±14 Dalton, about 7024±13 Dalton, about 5382±97 Dalton and about 4475±81 Dalton; (iii) about 8141±15 Dalton, about 9149±16 Daltons, and about 9656±17 Dalton; (iv) about 9149±16 Dalton and about 9508±17 Dalton; (v) about 5074±91 Dalton, about 9149±16 Dalton and about 9656±17 Dalton; or (vi) about 5382±97 Dalton, about 7024±13 Dalton and about 7820±14 Dalton; and (b) second computer program code means for correlating the determination to a diagnosis of benign prostate hyperplasia, prostate cancer or a negative diagnosis.

In yet another form of the invention, a computer readable medium may include (a) first computer program code means for detecting at least two protein biomarkers in a test sample from a subject, said protein biomarkers having a molecular weight selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons; and (b) second computer program means for correlating the detection to a diagnosis of benign prostate hyperplasia, prostate cancer or a negative diagnosis.

It is an object of the present invention to provide methods to diagnose, or aid in the diagnosis of, prostate cancer or benign prostate hyperplasia, or to otherwise make a negative diagnosis.

It is a further object of the invention to provide kits that may be utilized to detect the biomarkers described herein and that may be utilized to diagnose, or aid in the diagnosis of, prostate cancer or benign prostate hyperplasia.

It is another object to provide methods of using a plurality of classifiers to make a probable diagnosis of prostate cancer or benign prostate hyperplasia.

It is a further object of the invention to provide computer program media storing computer instructions therein for instructing a computer to perform a computer-implemented process for developing and/or using a plurality of classifiers to make a probable diagnosis of prostate cancer, benign prostate hyperplasia, or a negative diagnosis.

These and other objects and advantages of the present invention will be apparent from the descriptions herein.

### BRIEF DESCRIPTION OF THE FIGURES

FIG. 1 shows a flow diagram that summarizes the process from peak detection to sample classification as more fully described in Example 1.

FIG. 2A depicts a schematic of the decision tree classification system utilized in example 1. FIG. 2B depicts the SELDI protein profiles showing various features of the classification system. FIG. 2C depicts the SELDI protein profiles obtained after storing samples for a prolonged period of time.

FIG. 3 depicts representative raw spectra of peaks resolved between 2000-40000 Daltons utilizing SELDI as more fully described in Example 2. The top spectra represents spectra of peaks resolved having a molecular weight in the range of about 2000 to about 10000 Daltons; the bottom spectra represents spectra of peaks resolved having a molecular weight of about 10000 to about 40000 Daltons.

FIG. 4 depicts graphs showing the training error rate minimal margin and/or test error rate for the boosted decision tree classifier described in Example 2. FIG. 4A depicts the training error rate, the minimal margin, and the generalization error rate (testing error) of M, the number of base stumps for the boosted decision tree classifier distinguishing between non-cancer and cancer. After the training error reaches zero (on round 47), the minimal margin keeps increasing, and the generalization error keeps decreasing, finally reaching zero (on round 265); FIG. 4B depicts the training error rate and the minimal margin against the number of base stumps for the boosted decision tree classifier distinguishing between normal and BPH. After the training error reaches zero (on round 9), the minimal margin keeps increasing.

FIG. 5 illustrates one example of a central processing unit for implementing a computer process in accordance with a computer implemented embodiment of the present invention.

FIG. 6 illustrates one example of a block diagram of internal hardware of the central processing unit of FIG. 5.

FIG. 7 is an illustrative computer-readable medium upon which computer instructions can be embodied.

### DESCRIPTION OF THE PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention relates to methods for aiding in a diagnosis of, and methods for diagnosing, benign prostate hyperplasia and prostate cancer. Surface enhanced laser desorption/ionization mass spectroscopy has been combined with various algorithms to deduce protein biomarkers that may be utilized in various decision trees to aid in the diagnosis of, and/or to diagnose, benign prostate hyperplasia, prostate cancer or to make a negative diagnosis.

The methods of the present invention effectively differentiate between individuals with benign prostate hyperplasia, prostate cancer or normal individuals. As defined herein, normal individuals are individuals with a negative diagnosis with respect to benign prostate hyperplasia or prostate cancer. That is, normal individuals do not have benign prostate hyperplasia or prostate cancer. The method includes detecting a protein biomarker in a test sample from a subject. For example, the protein biomarkers having a molecular weight of about 4475±81, about 5074±91, about 5382±97, about 7024±13, about 7820±14, about 8141±15, about 9149±16, about 9508±17, and about 9656±17 Daltons have been identified that aid in the probable diagnosis of benign prostate hyperplasia, prostate cancer or aid in a negative diagnosis. In addition, the protein biomarkers having a molecular weight of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons have also been identified to aid in the diagnosis. In accordance with the present invention, at least two or more of the protein biomarkers are detected. As used herein, the term “detecting” includes determining the presence, the absence, the quantity, or a combination thereof, of the protein biomarkers. The quantity of the biomarkers may be represented by the peak intensity as identified by mass spectrometry, for example, or concentration of the biomarkers.

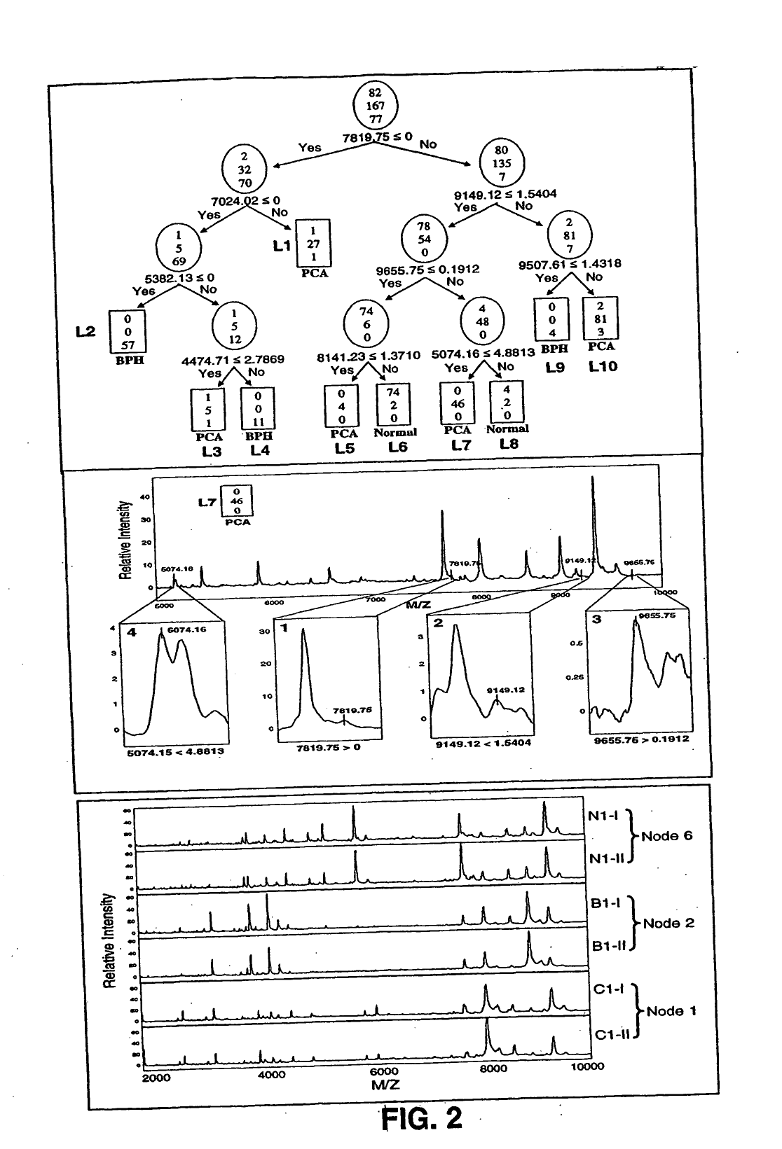

In certain forms of the invention, selected groups of protein biomarkers find utility in diagnosing prostate cancer or benign prostate hyperplasia. For example, the following groups of markers find utility in making, or otherwise aiding in making, a specific diagnosis: (1) 7820 and 7024 Dalton biomarkers to diagnose prostate cancer; (2) 7820, 7024 and 5382 Dalton biomarker to diagnose benign prostate hyperplasia; (3) the 7820, 7024, 5382 and 4474 Dalton biomarkers to distinguish between prostate cancer and benign prostate hyperplasia; (4) the 9149 and 9508 Dalton biomarkers to distinguish between prostate cancer and benign prostate hyperplasia; (5) the 9149, 9656 and 8141 Dalton biomarkers to distinguish between prostate cancer and normal individuals; and (6) the 9149, 9656 and 5074 Dalton biomarkers to distinguish between individuals with prostate cancer and normal individuals. The decision tree showing, for example, how such markers may be utilized is shown in FIG. 2.

As discussed above, the presence, absence and/or quantity of the various biomarkers may be utilized to make, or otherwise aid in making, a specific diagnosis. For example, and referring to FIG. 2, the absence of the 7820 peak and the presence of the 7024 peak may be correlated to a diagnosis of prostate cancer. Referring now to the second group above, the absence of the 5382, 7024 and 7820 Dalton biomarkers may be correlated to benign prostate hyperplasia.

The presence and absence of selected biomarkers, along with the quantity of other biomarkers, may also be utilized to make, or otherwise aid in making, a specific diagnosis. For example, referring to group 3 mentioned above, the absence of the 7820 Dalton and the 7024 Dalton biomarkers, the presence of the 5382 Dalton biomarker and the presence of the 4475 Dalton biomarker below the indicated threshold value in FIG. 2 may be correlated to a diagnosis of prostate cancer, whereas if the 4475 Dalton biomarker is present in this same group in a quantity above the threshold value, a negative diagnosis may be made. The threshold values in FIG. 2 represent the normalized peak intensity of the biomarkers. As more fully described in Example 1, these threshold values may represent the normalized peak intensity of a particular biomarker or the concentration of the biomarker. The normalization process may involve subtracting out the ion current not related to the proteins analyzed. The normalization process could alternatively involve reporting the peak intensity relative to the peak intensity of an internal or external control. For example, a known protein may be added to the system. Additionally, a known protein produced by the test subject, such as albumin, may act as an internal standard or control.

Turning now to the next group 4, presence of the 9149 and the 9508 Dalton peaks above the indicated threshold values in FIG. 2 correlate to a diagnosis of prostate cancer whereas presence of the 9149 Dalton biomarker above the indicated threshold value and the 9508 Dalton biomarker below the indicated threshold value may be correlated to a diagnosis of benign prostate hyperplasia.

Turning now to group 5, if the 9149, 9655 and 8141 Dalton biomarkers are below the indicated threshold values in FIG. 2, a correlation to prostate cancer may be made. Alternatively, if the 9149 and 9655 Dalton biomarkers are below the indicated threshold values, and the 8141 Dalton biomarker is above the indicated threshold value, a negative diagnosis may be made.

Turning now to group 6, the presence of the 9656 Dalton biomarker above a specified threshold value indicated in FIG. 2, and the presence of the 9149 and 5074 Dalton biomarkers below indicated threshold values, may be correlated to a diagnosis of prostate cancer. Alternatively, the presence of the 9656 and 5074 Dalton biomarkers above the indicated threshold values indicated in FIG. 2, and the presence of the 9149 Dalton biomarker below the indicated threshold value may be correlated to a negative diagnosis.

In yet another form of the method described above, the protein biomarkers that may be detected include those having a molecular weight selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons. Correlation of the detection of these biomarkers with prostate cancer, benign prostate hyperplasia, or a negative diagnosis is preferably accomplished utilizing a boosted decision tree analysis as more fully described in Example 2.

The method includes detecting at least one protein biomarker. However, any number of biomarkers may be detected. It is preferred that at least two protein biomarkers are detected in the analysis. However, it is realized that three, four, or more, including all, of the biomarkers described herein may be utilized in the diagnosis. Thus, not only can one or more markers be detected, one to nine, preferably two to nine, two to twelve and two to twenty-one biomarkers, or some other combination, may be detected and analyzed as described herein. In addition, other protein biomarkers not herein described may be combined with any of the presently disclosed protein biomarkers to aid in the diagnosis of prostate cancer or benign prostate hyperplasia. Moreover, any combination of the above protein biomarkers may be detected in accordance with the present invention. The inventors have found that selected groups of the protein biomarkers find utility in diagnosing prostate cancer or benign prostate hyperplasia. For example, the protein biomarkers having a molecular weight selected from the group consisting of about 3963±7, about 4079±7, about 6542±12, about 6797±12, about 6949±13; about 6990±13, about 7024±13, about 7885±14, about 8067±15, about 8356±15, about 9656±17, about 9720±18 Daltons may advantageously be utilized for diagnosing prostate cancer from a negative diagnosis. Additionally, the protein biomarkers having a molecular weight selected from the group consisting of about 3486±6, about 4071±7, about 4580±8, about 5298±10, about 6099±11, about 7054±13, about 7820±14, about 7844±14, and about 8943±16 may advantageously be utilized to distinguish benign prostate hyperplasia from a negative diagnosis.

The detection of the protein biomarkers described herein in a test sample may be performed in a variety of ways. In one form of the invention, a method for detecting the biomarker includes detecting the biomarker by gas phase ion spectrometry utilizing a gas phase ion spectrometer. The method may include contacting a test sample having a biomarker, such as the protein biomarkers described herein, with a substrate comprising an adsorbent thereon under conditions to allow binding between the biomarker and the adsorbent and detecting the biomarker bound to the adsorbent by gas phase ion spectrometry.

A wide variety of adsorbents may be used. The adsorbents may include a hydrophobic group, a hydrophilic group, a cationic group, an anionic group, a metal ion chelating group, or antibodies which specifically bind to an antigenic biomarker, or some combination thereof. (such as a “mixed mode” adsorbent). Exemplary adsorbents that include a hydrophobic group include matrices having aliphatic hydrocarbons, such as C₁-C₁₈ aliphatic hydrocarbons and matrices having aromatic hydrocarbon functional groups, including phenyl groups. Exemplary adsorbents that include a hydrophilic group include silicon oxide, or hydrophilic polymers such as polyalkylene glycol, such as polyethylene glycol; dextran, agarose or cellulose. Exemplary adsorbents that include a cationic group include matrices of secondary, tertiary or quaternary amines. Exemplary adsorbents that have an anionic group include matrices of sulfate anions and matrices of carboxylate anions or phosphate anions. Exemplary adsorbents that have metal chelating groups include organic molecules that have one or more electron donor groups which may form coordinate covalent bonds with metal ions, such as copper, nickel, cobalt, zinc, iron, aluminum and calcium. Exemplary adsorbents that include an antibody include antibodies that are specific for any of the biomarkers provided herein and may be readily made by methods known to the skilled artisan.

Alternatively, the substrate can be in the form of a probe which may be removably insertable into a gas phase ion spectrometer. For example, a substrate may be in the form of a strip with adsorbents on its surface. In yet other forms of the invention, the substrate can be positioned onto a second substrate to form a probe which may be removably insertable into a gas phase ion spectrometer. For example, the substrate can be in the form of a solid phase, such as a polymeric or glass bead with a functional group for binding the marker, which can be positioned on a second substrate to form a probe. The second substrate may be in the form of a strip, or a plate having a series of wells at predetermined locations. In this manner, the biomarker can be adsorbed to the first substrate and transferred to the second substrate which can then be submitted for analysis by gas phase ion spectrometry.

The probe can be in the form of a wide variety of desired shapes, including circular, elliptical, square, rectangular, or other polygonal or other desired shape, as long as it is removably insertable into a gas phase ion spectrometer. The probe is also preferably adapted or otherwise configured for use with inlet systems and detectors of a gas phase ion spectrometer. For example, the probe can be adapted for mounting in a horizontally and/or vertically translatable carriage that horizontally and/or vertically moves the probe to a successive position without requiring, for example, manual repositioning of the probe.

The substrate that forms the probe can be made from a wide variety of materials that can support various adsorbents. Exemplary materials include insulating materials, such as glass and ceramic; semi-insulating materials, such as silicon wafers; electrically-conducting materials (including metals such as nickel, brass, steel, aluminum, gold or electrically-conductive polymers); organic polymers; biopolymers, or combinations thereof.

In other embodiments of the invention, depending on the nature of the substrate, the substrate surface may form the adsorbent. In other cases, the substrate surface may be modified to incorporate thereon a desired adsorbent. The surface of the substrate forming the probe can be treated or otherwise conditioned to bind adsorbents that may bind markers if the substrate can not bind biomarkers by itself. Alternatively, the surface of the substrate can also be treated or otherwise conditioned to increase its natural ability to bind desired biomarkers. Other probes suitable for use in the invention may be found, for example, in PCT international publication numbers WO 01/25791 (Tai-Tung et al.) and WO 01/71360 (Wright et al.).

The adsorbents may be placed on the probe substrate in a wide variety of patterns, including a continuous or discontinuous pattern. A single type of adsorbent, or more than one type of adsorbent, may be placed on the substrate surface. The patterns may be in the form of lines, curves, such as circles, or other shape or pattern as desired and as known in the art.

The method of production of the probes will depend on the selection of substrate materials and/or adsorbents as known in the art. For example, if the substrate is a metal, the surface may be prepared depending on the adsorbent to be applied thereon. For example, the substrate surface may be coated with a material, such as silicon oxide, titanium oxide or gold, that allows derivatization of the metal surface to form the adsorbent. The substrate surface may then be derivatized with a bifunctional linker, one of which binds, such as covalently binds, with a functional group on the surface and the opposing end of the linker may be further derivatized with groups that function as an adsorbent. As a further example, a substrate that includes a porous silicon surface generated from crystalline silicon can be chemically modified to include adsorbents for binding markers. Additionally, adsorbents with a hydrogel backbone can be formed directly on the substrate surface by in situ polymerization of a monomer solution which includes, for example, substituted acrylamide or acrylate monomers, or derivatives thereof that include a functional group of choice as adsorbent.

In preferred forms of the invention, the probe may be a chip, such as those available from Ciphergen Biosystems, Inc. (Palo Alto, Calif.). The chip may be a hydrophilic, hydrophobic, anion-exchange, cation-exchange, immobilized metal affinity or preactivated protein chip array. The hydrophobic chip may be a ProteinChip H4, which includes a long-chain aliphatic surface that binds proteins by reverse phase interaction. The hydrophilic chip may be ProteinChips NP1 and NP2 which include a silicon dioxide substrate surface. The cation exchange ProteinChip array may be ProteinChip WCX2, a weak cation exchange array with a carboxylate surface to bind cationic proteins. Alternatively, the chip may be an anion exchange protein chip array, such as SAX1 (strong anion exchange) ProteinChip which is made from silicon-dioxide-coated aluminum substrates, or ProteinChip SAX2 with a higher capacity quaternary ammonium surface to bind anionic proteins. A further useful chip may be the immobilized metal affinity capture chip (IMAC3) having nitrilotriacetic acid on the surface. Further alternatively, ProteinChip PS1 is available which includes a carbonyldiimidazole surface which covalently reacts with amino groups or may be ProteinChip PS2 which includes an epoxy surface which covalently reacts with amine and thiol groups.

In accordance with the present invention, the probe contacts a test sample. The test sample may be obtained from a wide variety of sources. The sample is typically obtained from biological fluid from a subject or patient who is being tested for prostate cancer, benign prostate hyperplasia or from a normal individual, or who is otherwise thought to be at risk for such diseases. A preferred biological fluid is blood or blood sera. Other biological fluids in which the biomarkers may be found include, for example, seminal fluid, seminal plasma, saliva, lymph fluid, lung/bronchial washes, mucus, nipple secretions, sputum, tears and saliva. Other test sample sources include, for example, feces. If necessary, the sample can be solubilized in or mixed with an eluant prior to being contacted with the probe. The probe may contact the test sample solution by a wide variety of techniques, including bathing, soaking, dipping, spraying, washing, pipetting or other desirable methods. The method is performed so that the adsorbent of the probe preferably contacts the test sample solution. Although the concentration of the biomarker or biomarkers in the sample may vary, it is generally desirable to contact a volume of test sample that include about 1 altomole to about 100 picomoles of marker in about 1 μl to about 500 μl solution for binding to the adsorbent.

The sample and probe contact each other for a period of time sufficient to allow the biomarker to bind to the adsorbent. Although this time may vary depending on the nature of the sample, the nature of the biomarker, the nature of the adsorbent and the nature of the solution the biomarker is dissolved in, the sample and adsorbent are typically contacted for a period of about 30 seconds to about 12 hours, preferably about 30 seconds to about 15 minutes.

The temperature at which the probe contacts the sample will depend on the nature of the sample, the nature of the sample, the nature of the biomarker, the nature of the adsorbent and the nature of the solution the biomarker is dissolved in. Generally, the sample may be contacted with the probe under ambient temperature and pressure and conditions. However, the temperature and pressure may vary as desired. For example, the temperature may vary from about 4° C. to about 37° C.

After the sample has contacted the probe for a period of time sufficient for the marker to bind to the adsorbent or substrate surface should no adsorbent be used, unbound material may be washed from the substrate or adsorbent surface so that only bound materials remain on the respective surface. The washing can be accomplished by, for example, bathing, soaking dipping, rinsing, spraying or otherwise washing the respective surface with an eluant or other washing solution. A microfluidics process is preferably used when a washing solution such as an eluant is introduced to small spots of adsorbents on the probe. The temperature of the washing solution may vary, but is typically about 0° C. to about 100° C., and preferably about 4° C. and about 37° C.

A wide variety of washing solutions may be utilized to wash the probe substrate surface. The washing solutions may be organic solutions or aqueous solutions. Exemplary aqueous solutions may be buffered solutions, including HEPES buffer, a Tris buffer, phosphate buffered saline or other similar buffers known to the art. The selection of a particular washing solution will depend on the nature of the biomarkers and the nature of the adsorbent utilized. For example, if the probe includes a hydrophobic group and a sulfonate group as adsorbents, such as the SCXI PorteinChip® array, then an aqueous solution, such as a HEPES buffer, may be used. As a further example, if a probe includes a metal binding group as an adsorbent, such as with the Ni(II) ProteinChip® array, than an aqueous solution, such as a phosphate buffered saline may be preferred. As yet a further example, if a probe includes a hydrophobic group as an adsorbent, such as with the HF ProteinChip® array, water may be a preferred washing solution.

An energy absorbing molecule, such as one in solution, may be applied to the markers or other substances bound on the substrate surface of the probe. As used herein, an “energy absorbing molecule” refers to a molecule that absorbs energy from an energy source in a gas phase ion spectrometer, which may assist the desorption of markers or other substances from the surface of the probe. Exemplary energy absorbing molecules include cinnamic acid derivatives, sinapinic acid, dihyroxybenzoic acid and other similar molecules known to the art. The energy absorbing molecule may be applied by a wide variety of techniques previously discussed herein for contacting the sample and probe substrate, including, for example, spraying, pipetting or dipping, preferably after the unbound materials are washed off the probe substrate surface.

In another embodiment, the chip can be a SEND chip. “Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”). SEND biochips avoid the necessity of applying external matrix to the chip before laser desorption/ionization.

After the biomarker is appropriately bound to the probe, the biomarker may be detected, quantified and/or its characteristics may be otherwise determined using a gas phase ion spectrometer. As known in the art, gas phase ion spectrometers include, for example, mass spectrometers, ion mobility spectrometers, and total ion current measuring devices.

In a preferred embodiment, a mass spectrometer is utilized to detect the biomarkers bound to the substrate surface of the probe. The probe, with the bound marker on its surface, may be introduced into an inlet system of the mass spectrometer. The marker may then be ionized by an ionization source, such as a laser, fast atom bombardment, plasma or other suitable ionization sources known to the art. The generated ions are typically collected by an ion optic assembly and a mass analyzer then disperses and analyzes the passing ions. The ions exiting the mass analyzer are detected by a detector. The detector translates information of the detected ions into mass-to-charge ratios. Detection and/or quantitation of the marker will typically involve detection of signal intensity.

In further preferred forms of the invention, the mass spectrometer is a laser desorption time-of-flight mass spectrometer, and further preferably surface enhanced laser desorption time-f-flight mass spectrometry (SELDI) is utilized. SELDI is an improved method of gas phase ion spectrometry for biomolecules. In SELDI, the surface on which the analyte is applied plays an active role in the analyte capture, and/or desorption.

As known in the art, in laser desorption mass spectrometry, a probe with a bound marker is introduced into an inlet system. The marker is desorbed and ionized into the gas phase by a laser ionization source. The ions generated are collected by an ion optic assembly. Ions are accelerated in a time-of-flight mass analyzer through a short high voltage field and allowed to drift into a high vacuum chamber. The accelerated ions strike a sensitive detector surface at a far end of the high vacuum chamber at a different time. As the time-of-flight is a function of the mass of the ions, the elapsed time between ionization and impact can be used to identify the presence or absence of molecules of specific mass. Quantitation of the biomarkers, either in relative or absolute amounts, may be accomplished by comparison of the intensity of the displayed signal of the biomarker to a control amount of a biomarker or other standard as known in the art. The components of the laser desorption time-of-flight mass spectrometer may be combined with other components described herein and/or known to the skilled artisan that employ various means of desorption, acceleration, detection, or measurement of time.

In further embodiments, detection and/or quantitation of the biomarkers may be accomplished by matrix-assisted laser desorption ionization (MALDI). MALDI also provides for vaporization and ionization of biological samples from a solid-state phase directly into the gas phase. As known in the art, the sample, including the desired analyte, is dissolved or otherwise suspended in, a matrix that co-crystallizes with the analyte, preferably to prevent the degradation of the analyte during the process.

In another form of the invention, an ion mobility spectrometer can be used to detect and characterize the biomarkers described herein. The principle of ion mobility spectrometry is based on different mobility of ions. Specifically, ions of a sample produced by ionization move at different rates, due to their difference in, for example, mass, charge, or shape, through a tube under the influence of an electric field. The ions (typically in the form of a current) are registered at the detector which can then be used to identify a marker or other substances in the sample. One advantage of ion mobility spectrometry is that it can operate at atmospheric pressure.

In another embodiment, a total ion current measuring device can be used to detect and characterize the biomarkers described herein. This device can be used, for example, when the probe has a surface chemistry that allows only a single type of marker to be bound. When a single type of marker is bound on the probe, the total current generated from the ionized biomarker reflects the nature of the marker. The total ion current produced by the biomarker can then be compared to stored total ion current of known compounds. Characteristics of the biomarker can then be determined.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

Data generated by desorption and detection of the biomarkers can be analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. Using this information, the program can then identify the set of features on the probe defining certain selectivity characteristics, such as types of adsorbent and eluants used. The computer also contains code that receives data on the strength of the signal at various molecular masses received from a particular addressable location on the probe as input. This data can indicate the number of biomarkers detected, optionally including the strength of the signal and the determined molecular mass for each biomarker detected.

Data analysis can include the steps of determining signal strength (e.g., height of peaks, area of peaks) of a biomarker detected and removing “outerliers” (data deviating from a predetermined statistical distribution). For example, as previously mentioned, the observed peaks can be normalized, a process whereby the height of each peak relative to some reference is calculated. For example, a reference can be background noise generated by instrument and chemicals (e.g., energy absorbing molecule) which is set as zero in the scale. The signal strength can then be detected for each biomarker or other substances can be displayed in the form of relative intensities in the scale desired (e.g., 100). Alternatively, a standard may be included with the sample so that a peak from the standard can be used as a reference to calculate relative intensities of the signals observed for each biomarker or other markers detected as previously discussed.

The computer can transform the resulting data into various formats for displaying. In one format, referred to as “spectrum view or retentate map,” a standard spectral view can be displayed, wherein the view depicts the quantity of biomarker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be convened into a grayscale image based on the height of each peak, resulting in an appearance similar to bands on electrophoretic gels. In a further format, referred to as “3-D overlays,” several spectra can be overlayed to study subtle changes in relative peak heights. In yet a further format, referred to as “difference map view,” two or more spectra can be compared, conveniently highlighting unique biomarkers and biomarkers which are up- or down-regulated between samples. Biomarker profiles (spectra) from any two samples may be compared visually.

Using any of the above display formats, it can be readily determined from the signal display whether a biomarker having a particular molecular weight is detected from a sample. Moreover, from the strength of signals, the amount of markers bound on the probe surface can be determined.

In preferred forms of the invention, a single decision tree classification algorithm is utilized to analyze the data generated from SELDI. Such an algorithm is more specifically described in example 1 herein. In more preferred forms of the invention, the a boosted decision tree algorithm is utilized to analyze the data generated from SELDI. Such an algorithm is more specifically described in, for example, example 2. Such a process results in improved specificity and selectivity as more fully described in Example 2.

The test samples may be pre-treated prior to being subject to gas phase ion spectrometry. For example, the samples can be purified or otherwise pre-fractionated to provide a less complex sample for analysis. The optional purification procedure for the biomolecules present in the test sample may be based on the properties of the biomolecules, such a size, charge and function. Methods of purification include centrifugation, electrophoresis, chromatography, dialysis or a combination thereof. As known in the art, electrophoresis may be utilized to separate the biomolecules in the sample based on size and charge. Electrophoretic procedures are well known to the skilled artisan, and include isoelectric focusing, sodium dodecyl sulfate polyacrylamide gel electrophoresis (SDS-PAGE), agaroase gel electrophoresis, and other known methods of electrophoresis.

The purification step may be accomplished by a chromatographic fractionation technique, including size fractionation, fractionation by charge and fractionation by other properties of the biomolecules being separated. As known in the art, chromatographic systems include a stationary phase and a mobile phase, and the separation is based upon the interaction of the biomolecules to be separated with the different phases. In preferred forms of the invention, column chromatographic procedures may be utilized. Such procedures include partition chromatography, adsorption chromatography, size-exclusion chromatography, ion-exchange chromatography and affinity chromatography. Such methods are well known to the skilled artisan. In size exclusion chromatography, it is preferred that the size fractionation columns exclude molecules whose molecular mass is greater than about 10,000 Da.

In a preferred form of the invention, the sample is purified or otherwise fractionated on a bio-chromatographic chip by retentate chromatography before gas phase ion spectrometry. A preferred chip is the Protein Chip™ available from Ciphergen Biosystems, Inc. (Palo Alto, Calif.). As described above, the chip or probe is adapted for use in a mass spectrometer. The chip comprises an adsorbent attached to its surface. This adsorbent can function, in certain applications, as an in situ chromatography resin. In operation, the sample is applied to the adsorbent in an eluant solution. Molecules for which the adsorbent has affinity under the wash condition bind to the adsorbent. Molecules that do not bind to the adsorbent are removed with the wash. The adsorbent can be further washed under various levels of stringency so that analytes are retained or eluted to an appropriate level for analysis. An energy absorbing molecule can then be added to the adsorbent spot to further facilitate desorption and ionization. The analyte is detected by desorption from the adsorbent, ionization and direct detection by a detector. Thus, retentate chromatography differs from traditional chromatography in that the analyte retained by the affinity material is detected, whereas in traditional chromatography, material that is eluted from the affinity material is detected.

In yet another form of the invention, the biomarkers of the present invention may be detected, qualitatively or quantitatively, by an immunoassay procedure. The immunoassay typically includes contacting a test sample with an antibody that specifically binds to or otherwise recognizes a biomarker, and detecting the presence of a complex of the antibody bound to the biomarker in the sample. The immunoassay procedure may be selected from a wide variety of immunoassay procedures known to the art involving recognition of antibody/antigen complexes, including enzyme immunassays, competitive or non-competitive, and including enzyme-linked immunosorbent assays (ELISA), radioimmunoassays (RIA), and Western blots, and use of multiplex assays, including use of antibody arrays wherein several desired antibodies are placed on a support, such as a glass bead or plate, and reacted or otherwise contacted with the test sample. Such assays are well known to the skilled artisan and are described, for example, more thoroughly in Antibodies: A Laboratory Manual (1988) by Harlow & Lane; Immunoassays: A Practical Approach, Oxford University Press, Gosling, J. P. (ed.) (2001) and/or Current Protocols in Molecular Biology (Ausubel et al.) which is regularly and periodically updated.

The antibodies to be used in the immunoassays described herein may be polyclonal antibodies and may be obtained by procedures which are well known to the skilled artisan, including injecting purified biomarkers into various animals and isolating the antibodies produced in the blood serum. The antibodies may be monoclonal antibodies whose method of production is well known to the art, including injecting purified biomarkers into a mouse, for example, isolating the spleen cells producing the anti-serum, fusing the cells with tumor cells to form hybridomas and screening the hybridomas. The biomarkers may first be purified by techniques similarly well known to the skilled artisan, including the chromatographic, electrophoretic and centrifugation techniques described previously herein. Such procedures may take advantage of the protein biomarker's size, charge, solubility, affinity for binding to selected components, combinations thereof, or other characteristics or properties of the protein. Such methods are known to the art and can be found, for example, in Current Protocols in Protein Science, J. Wiley and Sons, New York, N.Y., Coligan et al. (Eds.) (2002; Harris, E. L. V., and S. Angal in Protein purification applications: a practical approach, Oxford University Press, New York, N.Y. (1990). Once the antibody is provided, a biomarker can be detected and/or quantitated by the immunoassays previously described herein.

Although specific procedures for immunoassays are well known to the skilled artisan, generally, an immunoassay may be performed by initially obtaining a sample as previously described herein from a test subject. The antibody may be fixed to a solid support prior to contacting the antibody with a test sample to facilitate washing and subsequent isolation of the antibody/protein biomarker complex. Examples of solid supports are well known to the skilled artisn and include, for example, glass or plastic in the form of, for example, a microtiter plate. Antibodies can also be attached to the probe substrate, such as the ProteinChip™ arrays described herein.

After incubating the test sample with the antibody, the mixture is washed and the antibody-marker complex may be detected. The detection can be accomplished by incubating the washed mixture with a detection reagent, and observing, for example, development of a color or other indicator. The detection reagent may be, for example, a second antibody which is labeled with a detectable label. Exemplary detectable labels include magnetic beads (e.g., DYNABEADS™), fluorescent dyes, radiolabels, enzymes (e.g., horseradish peroxide, alkaline phosphatase and others commonly used in enzyme immunoassay procedures), and colorimetric labels such as colloidal gold, colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the biomarker is incubated simultaneously with the mixture. The amount of an antibody-marker complex can be determined by comparing to a standard.

Throughout the assays, incubation and/or washing steps may be required after each combination of reagents. Incubation steps can vary from about 5 seconds to several hours, preferably from about 5 minutes to about 24 hours. However, the incubation time will depend upon the particular immunoassay, biomarker, and assay conditions. Usually the assays will be carried out at ambient temperature, although they can be conducted over a range of temperatures, such as about 0° C. to about 40° C.

In yet another aspect of the invention, kits are provided that may, for example, be utilized to detect the biomarkers described herein. The kits can, for example, be used to detect any one or more of the biomarkers described herein which may advantageously be utilized for diagnosing, or aiding in the diagnosis of, prostate cancer, benign prostate hyperplasia or in a negative diagnosis.

In one embodiment, a kit may include a substrate that includes an adsorbent thereon, wherein the adsorbent is preferably suitable for binding one or more protein biomarkers described herein, and instructions to detect the biomarker by contacting a test sample as described herein with the adsorbent and detecting the biomarker retained by the adsorbent. In certain embodiments, the kits may include an eluant, or instructions for making an eluant, wherein the combination of the eluant and the adsorbent allows detection of the protein biomarkers by, for example, use of gas phase ion spectrometry. Such kits can be prepared from the materials described herein. In yet another embodiment, the kit may include a first substrate that includes an adsorbent thereon (e.g., a particle functionalized with an adsorbent) and a second substrate onto which the first substrate can be positioned to form a probe which is removably insertable into a gas phase ion spectrometer. In other embodiments, the kit may include a single substrate which is in the form of a removably insertable probe with adsorbents on the substrate. In yet another embodiment, the kit may further include a pre-fractionation spin column (e.g, K-30 size exclusion column).

The kit may further include instructions for suitable operating parameters in the form of a label or a separate insert. For example, the kit may have standard instructions informing a consumer or other individual how to wash the probe after a particular form of sample is contacted with the probe. As a further example, the kit may include instructions for pre-fractionating a sample to reduce the complexity of proteins in the sample.

In a further embodiment, a kit may include an antibody that specifically binds to the marker and a detection reagent. Such kits can be prepared from the materials described herein. The kit may further include pre-fractionation spin columns as described above, as well as instructions for suitable operating parameters in the form of a label or a separate insert.

In yet another aspect of the invention, methods of using a plurality of classifiers to make a probable diagnosis of prostate cancer, benign prostate hyperplasia or a negative diagnosis are provided. In one form of the invention, a method includes a) obtaining mass spectra from a plurality of samples from normal subjects, subjects diagnosed with prostate cancer, and subjects diagnosed with benign prostate hyperplasia; b) applying a boosted decision tree analysis to at least a portion of the mass spectra to obtain a plurality of weighted base classifiers, wherein the classifiers include a peak intensity value and an associated threshold value; and c) making a probable diagnosis of at least one of prostate cancer, benign prostate hyperplasia, and a negative diagnosis based on a linear combination of the plurality of weighted base classifiers. In certain forms of the invention, the method includes using the peak intensity value and the associated threshold value in linear combination to make a probable diagnosis of at least one of prostate cancer, benign prostate hyperplasia and a negative diagnosis. The preferred algorithm and data treatment is more fully described in Example 2.

### Computer Implementation

The techniques of the present invention may be implemented on a computing system 104 such as that depicted in FIG. 5. In this regard, FIG. 5 is an illustration of a computer system 104 which is also capable of implementing some or all of the computer processing in accordance with at least one computer implemented embodiment of the present invention.

Viewed externally, in FIG. 5, a computer system designated by reference numeral 104 has a computer portion 112 having drives 502 and 504, which are merely symbolic of a number of disk drives which might be accommodated by the computer system. Typically, these could include a floppy disk drive 502, a hard disk drive (not shown externally) and a CD ROM 504. The number and type of drives vary, typically with different computer configurations. Disk drives 502 and 504 are in fact optional, and for space considerations, are can be omitted from the computer system.

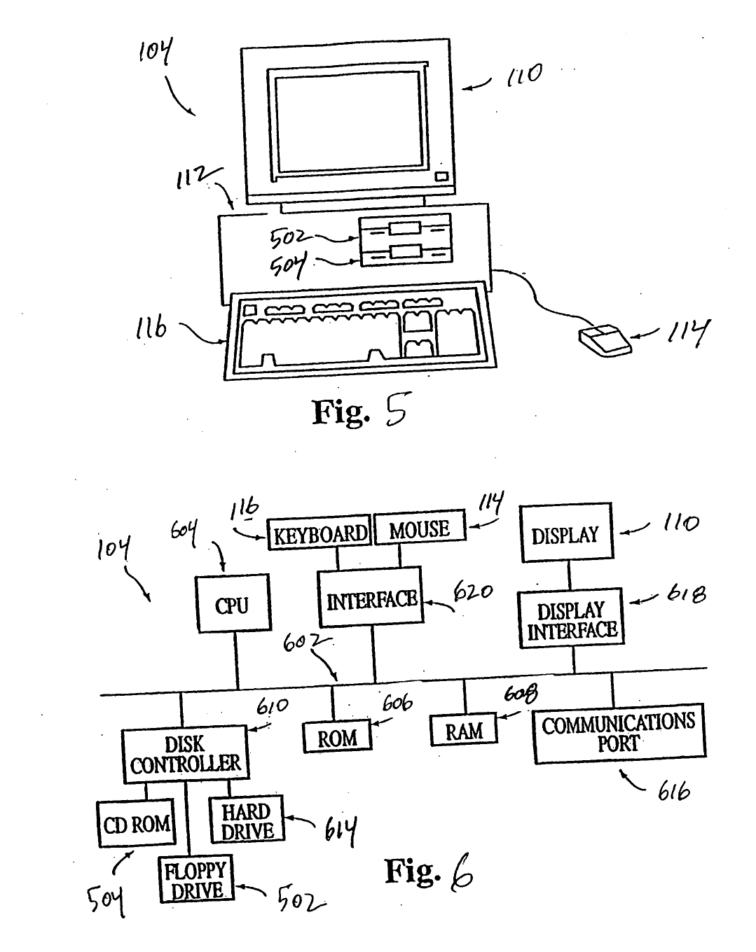

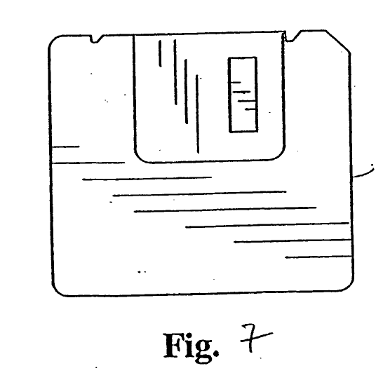

The computer system 104 also has an optional display monitor 110 upon which visual information pertaining to cells being normal or abnormal, suspected normal, suspected abnormal, etc. can be displayed. In some situations, a keyboard 116 and a mouse 114 are provided as input devices through which input may be provided, thus allowing input to interface with the central processing unit (CPU) 604 (FIG. 6). Then again, for enhanced portability, the keyboard 116 can be either a limited function keyboard or omitted in its entirety. In addition, mouse 114 optionally is a touch pad control device, or a track ball device, or even omitted in its entirety as well, and similarly may be used as an input device. In addition, the computer system 104 may also optionally include at least one infrared (or radio) transmitter and/or infrared (or radio) receiver for either transmitting and/or receiving infrared signals.

Although computer system 104 is illustrated having a single processor, a single hard disk drive 614 and a single local memory, the system 104 is optionally suitably equipped with any multitude or combination of processors or storage devices. Computer system 104 is, in point of fact, able to be replaced by, or combined with, any suitable processing system operative in accordance with the principles of the present invention, including hand-held, laptop/notebook, mini, mainframe and super computers, as well as processing system network combinations of the same.

FIG. 6 illustrates a block diagram of the internal hardware of the computer system 104 of FIG. 5. A bus 602 serves as the main information highway interconnecting the other components of the computer system 104. CPU 604 is the central processing unit of the system, performing calculations and logic operations required to execute a program. Read only memory (ROM) 606 and random access memory (RAM) 608 constitute the main memory of the computer system 104. Disk controller 610 interfaces one or more disk drives to the system bus 602. These disk drives are, for example, floppy disk drives such as 502, CD ROM or DVD (digital video disks) drive 504, or internal or external hard drives 614. As indicated previously, these various disk drives and disk controllers are optional devices.

A display interface 618 interfaces display 110 and permits information from the bus 602 to be displayed on the display 110. Again as indicated, display 110 is also an optional accessory. For example, display 110 could be substituted or omitted. Communications with external devices, for example, the other components of the system described herein, occur utilizing communication port 616. For example, optical fibers and/or electrical cables and/or conductors and/or optical communication (e.g., infrared, and the like) and/or wireless communication (e.g., radio frequency (RF), and the like) can be used as the transport medium between the external devices and communication port 616. Peripheral interface 620 interfaces the keyboard 116 and the mouse 114, permitting input data to be transmitted to the bus 602.

In alternate embodiments, the above-identified CPU 604, may be replaced by or combined with any other suitable processing circuits, including programmable logic devices, such as PALs (programmable array logic) and PLAs (programmable logic arrays). DSPs (digital signal processors), FPGAs (field programmable gate arrays), ASICs (application specific integrated circuits), VLSIs (very large scale integrated circuits) and the like.

Any presently available or future developed computer software language and/or hardware components can be employed in such embodiments of the present invention. For example, at least some of the functionality mentioned above could be implemented using Extensible Markup Language (XML), HTML, Visual Basic, C, C++, or any assembly language appropriate in view of the processor(s) being used. It could also be written in an interpretive environment such as Java and transported to multiple destinations to various users.

One of the implementations of the invention is as sets of instructions resident in the random access memory 608 of one or more computer systems 104 configured generally as described above. Until required by the computer system 104, the set of instructions may be stored in another computer readable memory, for example, in the hard disk drive 614, or in a removable memory such as an optical disk for eventual use in the CD-ROM 504 or in a floppy disk (e.g., floppy disk 702 of FIG. 7) for eventual use in a floppy disk drive 502. Further, the set of instructions (such as those written in Java, HTML, XML, Standard Generalized Markup Language (SGML), and/or Structured Query Language (SQL)) can be stored in the memory of another computer and transmitted via a transmission medium such as a local area network or a wide area network such as the Internet when desired by the user. One skilled in the art knows that storage or transmission of the computer program medium changes the medium electrically, magnetically, or chemically so that the medium carries computer readable information.

Any biomarker, individually, is useful in aiding in the determination of prostate cancer status. First, the selected biomarker is measured in a subject sample using the methods described herein, e.g., capture on a SELDI biochip followed by detection by mass spectrometry. Then, the measurement is compared with a diagnostic amount or control that distinguishes a prostate cancer status from a non-cancer status. The diagnostic amount will reflect the information herein that a particular biomarker is up-regulated or down-regulated in a cancer status compared with a non-cancer status. As is well understood in the art, the particular diagnostic amount used can be adjusted to increase sensitivity or specificity of the diagnostic assay depending on the preference of the diagnostician. The test amount as compared with the diagnostic amount thus indicates prostate cancer status.

While individual biomarkers are useful diagnostic markers, it has been found that a combination of biomarkers provides greater predictive value than single markers alone. Specifically, the detection of a plurality of markers in a sample increases the percentage of true positive and true negative diagnoses and would decrease the percentage of false positive or false negative diagnoses. Thus, preferred methods of the present invention comprise the measurement of more than one biomarker. The detection of the marker or markers is then correlated with a probable diagnosis of human cancer

In other embodiments, the measurement of markers can involve quantifying the markers to correlate the detection of markers with a probable diagnosis of cancer. Thus, if the amount of the markers detected in a subject being tested is different compared to a control amount (i.e., higher or lower than the control, depending on the marker), then the subject being tested has a higher probability of having cancer.

The correlation may take into account the amount of the marker or markers in the sample compared to a control amount of the marker or markers (up or down regulation of the marker or markers) (e.g., in normal subjects in whom human cancer is undetectable). A control can be, e.g., the average or median amount of marker present in comparable samples of normal subjects in whom human cancer is undetectable. The control amount is measured under the same or substantially similar experimental conditions as in measuring the test amount. The correlation may take into account the presence or absence of the markers in a test sample and the frequency of detection of the same markers in a control. The correlation may take into account both of such factors to facilitate determination of prostate cancer status.

In certain embodiments of the methods of qualifying cancer status, the methods further comprise managing subject treatment based on the status. As aforesaid, such management describes the actions of the physician or clinician subsequent to determining cancer status. For example, if the result of the methods of the present invention is inconclusive or there is reason that confirmation of status is necessary, the physician may order more tests. Alternatively, if the status indicates that surgery is appropriate, the physician may schedule the patient for surgery. In other instances, the patient may receive chemotherapy or radiation treatments, either in lieu of, or in addition to, surgery. Likewise, if the result is negative, e.g., the status indicates late stage cancer or if the status is otherwise acute, no further action may be warranted. Furthermore, if the results show that treatment has been successful, no further management may be necessary.

The invention also provides for such methods where the biomarkers (or specific combination of biomarkers) are measured again after subject management. In these cases, the methods are used to monitor the status of the cancer, e.g., response to cancer treatment, remission of the disease or progression of the disease. Because of the ease of use of the methods and the lack of invasiveness of the methods, the methods can be repeated after each treatment the patient receives. This allows the physician to follow the effectiveness of the course of treatment. If the results show that the treatment is not effective, the course of treatment can be altered accordingly. This enables the physician to be flexible in the treatment options.

In another example, the methods for detecting markers can be used to assay for and to identify compounds that modulate expression of these markers in vivo or in vitro.

The methods of the present invention have other applications as well. For example, the markers can be used to screen for compounds that modulate the expression of the markers in vitro or in vivo, which compounds in turn may be useful in treating or preventing cancer in patients.

In another example, the markers can be used to monitor the response to treatments for cancer.

In yet another example, the markers can be used in heredity studies to determine if the subject is at risk for developing cancer. For instance, certain markers may be genetically linked. This can be determined by, e.g., analyzing samples from a population of prostate cancer patients whose families have a history of prostate cancer. The results can then be compared with data obtained from, e.g., cancer patients whose families do not have a history of prostate cancer. The markers that are genetically linked may be used as a tool to determine if a subject whose family has a history of prostate cancer is pre-disposed to having prostate cancer.

Reference will now be made to specific examples illustrating the biomarkers, kits, computer program media and methods above. It is to be understood that the examples are provided to illustrate preferred embodiments and that no limitation to the scope of the invention is intended thereby.

### EXAMPLE 1

### Use of Serum Protein Fingerprinting and a Pattern Matching Algorithm for Diagnosing Prostate Cancer or Benign Prostate Hyperplasia

Materials and Methods

Serum Samples

Serum samples were obtained from the Virginia Prostate Center Tissue and Body Fluid Bank. The serum procurement, data management and blood collection protocols were approved by Eastern Virginia Medical School Institutional Review Board. Blood samples from patients diagnosed with either PCA or BPH were procured from the Department of Urology, Eastern Virginia Medical School, and the healthy men (HM) cohort was obtained from free screening clinics open to the general public. Only pre-treatment samples obtained at the time of diagnosis of PCA or BPH were used for this study. After informed consent, the sample was collected into a 10 cc Serum Separator Vacutainer Tube and after 30 minutes was centrifuged at 3750×100 rpm for 5 minutes. The serum was distributed into 500 ul aliquots, and stored frozen at −80° C. A quality control sample was prepared by pooling an equal amount of serum from each specimen of the age matched HM group, and storing 100 ul aliquots at −80° C. The quality control (QC) sample was used to determine reproducibility and as a control protein profile for each SELDI experiment.

### Patient and Donor Cohorts

Specimens from four groups of patients were used in this study: 96 age-matched HM (control), 92 benign prostate hyperplasia (BPH), 99 patients diagnosed with organ confined PCA (T1, T2) and 98 diagnosed with non-organ confined (T3, T4) PCA. A donor was selected for the HM if they had a normal digital rectal exam, a PSA <4.0 ng/ml, and had no evidence of prostatic disease. The HM group consisted of 48 Caucasian and 48 African American males ranging in age from 51-70 (mean of 60). There were 33 Caucasian, 2 African American, and 57 of unknown race in the BPH patients group, ranging in age from 48 to 86 (mean 67). The BPH patients were selected if they had PSA values between 4 and 10, low PSA velocities, and had multiple negative biopsies. The organ confined (T1, T2) PCA group consisted of 76 Caucasian, 20 African American, 1 Asian, and 2 of unknown race with ages ranging from 50 to 89 (mean 71). For the non-organ confined PCA group (T3, T4), there were 80 Caucasian, 16 African-American, and 2 of unknown race, ranging in age from 44 to 87 (mean 69). The range and mean PSA values for the HM group was 0.15-3.83 ng/ml (1.32 ng/ml); 0.0-10.91 ng/ml (4.60 ng/ml) for the BPH group; 0.0-95.16 ng/ml (10.10 ng/ml) for the PCA T1, T2 group; and 0.0-8752 ng/ml (206.93 ng/ml) for the PCA T3, T4 group.

### SELDI Protein Profiling

Various chip chemistries (hydrophobic, ionic, cationic, and metal binding) were initially evaluated to determine which affinity chemistry provided the best serum profiles in terms of number and resolution of proteins. The IMAC-Cu metal binding chip was observed to give the best results. IMAC-3 chips (Ciphergen Biosystems, Inc, Fremont, Calif.) were coated with 20 μl of 100 mM CuSO₄ on each array, placed on a TOMY Micro Tube Mixer (MT-360, Tomy Seiko Co., Ltd), and agitated for 5 minutes. The chips were rinsed with deionized (DI) water 10 times, 20 μl of 100 mM sodium acetate added to each array, and shaken for 5 minutes to remove the unbound copper. The chips were rinsed again with DI water (×10) and put into a bioprocessor (Ciphergen Biosystems, Inc.), which is a device to hold 12 chips and which allows for application of larger volumes of serum to each chip array. The bioprocessor was washed and shaken on a platform shaker at a speed of 250 rpm for 5 minutes with 200 μl PBS in each well. This was repeated twice more and each time the PBS buffer was discarded by inverting the bioprocessor on a paper towel. Serum samples for SELDI analysis were prepared by vortexing 20 μl of serum with 30 μl of 8M Urea/1% CHAPS in PBS in a 1.5 ml microfuge tube at 4° C. for 10 minutes. 100 μl of 1M urea with 0.125% CHAPS was added to the serum/Urea mixture and briefly vortexed. PBS was added to make a 1:5 dilution and placed on ice until applied to a protein chip array. 50 μl of the diluted serum/urea mixture was applied to each well, the bioprocessor sealed, and shaken on a platform shaker at a speed of 250 rpm for 30 minutes. The serum/urea mixture was discarded and the PBS washing step was repeated 3 times. The chips were removed from the bioprocessor, washed with DI water 10 times, air-dried and stored in the dark at room temperature until subjected to SELDI mass analysis. Prior to SELDI analysis, 0.5 ul of a saturated solution of the energy absorbing molecule (EAM) sinapinic acid in 50% (v/v) acetonitrile, 0.5% trifluoroacetic acid was applied onto each chip array twice, letting the array surface air dry between each sinapinic acid application. Chips were placed in the Protein Biological System II mass spectrometer reader (Ciphergen Biosystems, Inc.), and time of flight spectra were generated by averaging 192 laser shots collected in the positive mode at laser intensity 220, detector sensitivity 7, and focus lag time of 900 ns. Mass accuracy was calibrated externally using the All-in-1 peptide MW standard (Ciphergen).

### Data Analysis

The data analysis process used in this study involved three stages: (1) peak detection and alignment; (2) selection of peaks with the highest discrimatory power; and (3) data analysis using a Decision Tree algorithm. A stratified random sampling with 4 strata (PCA (T1/T2), PCA (T3/M1), BPH, HM) was used to separate the entire data set into training and test data sets prior to the analysis. The training data set consisted of SELDI spectra from 167 PCA, 77 BPH, and 82 normal serum samples. The validity and accuracy of the classification algorithm was then challenged with a blinded test data set consisting of 30 PCA, 15 BPH, and 15 normal samples.

### Peak Detection

Peak detection was performed using Ciphergen SELDI software versions 3.0 beta and 3.0 (Internet address: www.chiphergen.com). The mass range from 2000 to 40000 Da was selected for analysis because this range contained the majority of the resolved protein/peptides. The molecular masses from 0 to 2000 Da were eliminated from analysis because this area contains adducts and artifacts of the EAM and possibly other chemical contaminants. Peak detection involved (1) baseline subtraction, (2) mass accuracy calibration, (3) automatic peak detection, and (4) peak alignment determination. The software program calculates noise, peak area, and filter based on the criteria selected by the operator for data analysis. The settings used for this study were: fitting window width=100 data points; average noise=10 points; peak area was calculated using the slope based method; low minimum valley depth=10 times noise; high minimum valley depth=0.5 times noise; low and high sensitivity of peak height=10 and 2 times noise, respectively; auto peak detection slider=8 for mass range 2 to 4 KDa, 11 for mass range 4 to 8 KDa, and 8 for mass range 8 to 40 KD. An average of 80 peaks was detected in each spectrum.

### Peak Alignment

All the labeled peaks (total 63,157) from 772 spectra were exported from SELDI to an Excel spreadsheet. A PeakMiner algorithm (Internet address: www.evms.edu/vpc/seld), developed in-house, was used to sort all the peaks based on mass values from low to high mass. A mass error score, the measurement of mass difference between peak X and peak X+1, is calculated for each peak using (Mpx−Mpx+1)/Mpx, where Mpx is the mass value of peak X. For example, if the mass error score was less than 0.18%, peak X and peak X+1 would be aligned into one peak by averaging the mass values. If the mass error score was larger than 0.18%, peak X and peak X+1, would be considered two distinct peaks. This is an iterative process throughout all the labeled peaks to determine the alignment of peaks, and records all the samples with peak intensity corresponding to each peak mass.

### Feature Selection

The power of each peak in discriminating normal vs. PCA; normal vs. BPH; and BPH vs. PCA was determined by estimating the AUC, which ranges from 0.5 (no discriminating power) to 1.0 (complete separation).

### Decision Tree Classification

Construction of the decision tree classification algorithm was performed as described by Breiman et al. (14) with modifications (i.e., used a negative log likelihood as a criterion and used an AUC of 0.62 for data reduction to select 124 peaks from 779 peaks), using a training data set consisting of 326 samples (82 normal, 77 BPH, and 167 PCA). Classification trees split up a dataset into two bins or nodes, using one rule at a time in the form of a question. The splitting decision is defined by presence or absence and the intensity levels of one peak. For example, the answer to “does mass A have an intensity less than or equal to X” splits the data set into two nodes, a left node for yes and a right node for no. This “splitting” process continues until terminal nodes or leaves (L) are produced or further splitting has no gain. Classification of terminal nodes is determined by the group (“class”) of samples (i.e., PCA, BPH, Normal) representing the majority of samples in that node. A “cost” function is calculated that reflects the heterogeneity of each node: −log L=−ΣΣnj log(pj) where L is the likelihood of the multinomial distribution, nj is the number of samples in class j, and pj is the probability of class j. Peaks selected by this process to form the splitting rules are the ones that achieve the maximum reduction of cost in the two descendant nodes.

### Statistical Analyses

The AUC was computed to identify the peaks having the highest potential to discriminate the 3 groups, based on the probability that the test result from a diseased individual is more indicative of disease than that from a non-diseased individual (15). A Bayesian approach was used to calculate the expected probabilities of each class in each terminal node (16); and their 95% confidence intervals were calculated using the posterior Dirichlet distribution (16). The 95% confidence intervals were calculated by generating and sorting 4000 samples for the posterior Dirichlet distribution, and the 100^{th} and 3900^{th} sample considered as the lower and upper bounds of the 95% confidence intervals, respectively. Specificity was calculated as the ratio of the number of non-disease samples correctly classified to the total number of non-disease samples. Sensitivity was calculated at the ratio of the number of correctly classified diseased samples to the total number of diseased samples. The PPV was calculated by dividing the number of true PCA positives by the sum of the number of true PCA positives plus the number of false PCA positives. The NPV was calculated by dividing the number of true negative non-disease samples (BPH/HM) by the sum of the number of false negative plus the number of true negative non-disease samples (BPH/HM).

Results

Data Analysis

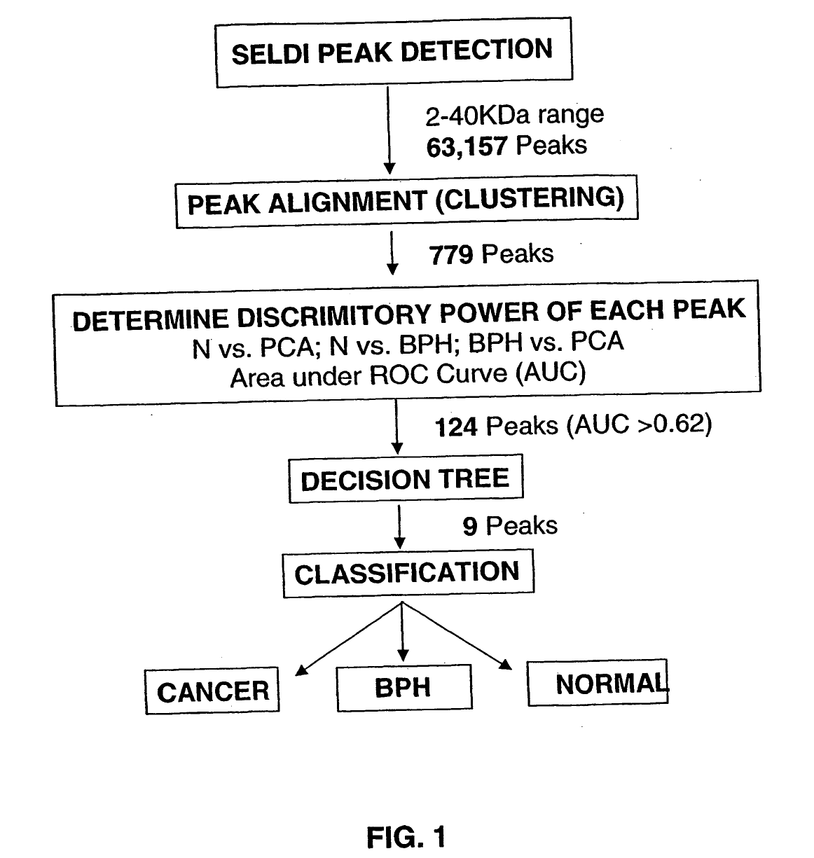

Peak detection using the SELDI software program detected 63,157 peaks in the 2-40 KDa mass range following analysis of 772 spectra (386 spectra in duplicate, with approximately 81 peaks/spectra). Of these, 779 peaks were identified following the clustering and peak alignment process. The AUC was calculated for each of the 779 peaks. No single peak was identified that has an AUC of 1.0, indicating that there was not a peak detected that alone could completely separate two groups (i.e. HM vs. PCA, or HM vs. BPH, or BPH vs. PCA) or three groups (PCA vs. BPH vs. HM). Of the 779 peaks, 124 had an AUC of equal to or higher than 0.62. Those with an AUC below 0.62 were considered irrelevant for classification. These 124 peaks identified in the training set were then used to construct the decision tree classification algorithm. FIG. 1 is a flow diagram that summarizes the process from peak detection to sample classification. The classification algorithm used 9 masses between 4-10 KDa to generate 10 terminal nodes (L1-L10) (FIG. 2A). Once the algorithm identifies the most discrimatory peaks, the classification rule is quite simple. For example, if an unknown sample has no peak at mass 7819.75 (“root” node) but has a peak at mass 7024.02, then the sample is placed in terminal node L1 and classified as PCA. If the sample is placed in L2, it will be assigned to BPH. Another example of this splitting process, is shown in FIG. 2B, in which 4 masses between 5-10 KDa are used to assign 46 of the 167 PCA samples to terminal node L7. Based on the stochastic nature of reality, misclassification of a new sample cannot be ruled out even for a pure node that contains only one sample type, for example L2 which contain only BPH samples. To obtain an idea if an unknown sample would be correctly classified or misclassified, the expected probability and 95% confidence level was calculated for each group in the 10 terminal nodes and is shown in the following Table 1:

TABLE 1
Expected probabilities and the ninety-five percent
confidence levels for each of the classes assigned to the ten
terminal nodes.

| Node | Class | Observation | Probability | 5% Confidence level |
|---|---|---|---|---|
| L1 | Normal | 1 | 0.0625 | 0.0081, 0.1693 |
| | PCA | 27 | 0.8750 | 0.7423, 0.9630 |
| | BPH | 1 | 0.0625 | 0.0087, 0.1698 |
| L2 | Normal | 0 | 0.0167 | 0.0005, 0.0584 |
| | PCA | 0 | 0.0167 | 0.0004, 0.0628 |
| | BPH | 57 | 0.9667 | 0.9072, 0.9952 |
| L3 | Normal | 1 | 0.2000 | 0.0247, 0.4793 |
| | PCA | 5 | 0.6000 | 0.3027, 0.8592 |
| | BPH | 1 | 0.2000 | 0.0248, 0.4753 |
| L4 | Normal | 0 | 0.0714 | 0.0018, 0.2509 |
| | PCA | 0 | 0.0714 | 0.0019, 0.2579 |
| | BPH | 11 | 0.8571 | 0.6311, 0.9823 |
| L5 | Normal | 0 | 0.1429 | 0.0040, 0.4725 |
| | PCA | 4 | 0.7143 | 0.3557, 0.9567 |
| | BPH | 0 | 0.1429 | 0.0040, 0.4504 |
| L6 | Normal | 74 | 0.9494 | 0.8950, 0.9858 |
| | PCA | 2 | 0.0380 | 0.0082, 0.0879 |
| | BPH | 0 | 0.0127 | 0.0003, 0.0459 |
| L7 | Normal | 0 | 0.0204 | 0.0005, 0.0738 |
| | PCA | 46 | 0.9592 | 0.8893, 0.9951 |
| | BPH | 0 | 0.0204 | 0.0005, 0.0726 |
| L8 | Normal | 4 | 0.5556 | 0.2458, 0.8416 |
| | PCA | 2 | 0.3333 | 0.0836, 0.6544 |
| | BPH | 0 | 0.1111 | 0.0032, 0.3732 |
| L9 | Normal | 0 | 0.1429 | 0.0034, 0.4560 |
| | PCA | 0 | 0.1429 | 0.0037, 0.4830 |
| | BPH | 4 | 0.7143 | 0.3354, 0.9566 |
| L10 | Normal | 2 | 0.0337 | 0.0068, 0.0784 |
| | PCA | 81 | 0.9213 | 0.8595, 0.9674 |
| | BPH | 3 | 0.0449 | 0.0123, 0.0964 |

The expected probabilities for HM and PCA samples to be misclassified in L2, for example, are 1.67%. Although not zero, the likelihood of HM or PCA samples being assigned to this node is extremely low; whereas BPH has a 96.67% chance of being correctly classified to L2 (with the 95% confidence interval between 90.72% and 99.52%). The probability of incorrect assignment of samples increases in nodes that contain either few majority samples or when only a few samples are assigned to the node, as for example terminal nodes L3, L5, and L9 (FIG. 2A). A summation of the classification results from the 10 terminal nodes is presented for the training and test sets in Table 2 seen below.

TABLE 2
Decision Tree Classification of the Prostate Training and Test Sets
A. Training Set

| | | | | |
|---|---|---|---|---|
| Sample | Normal | BPH | PCA | Misclassified Rate |

| | | | | | | | | |
|---|---|---|---|---|---|---|---|---|
| Normal | 78 | 95.12% | 0 | 0.00% | 4 | 4.88% | 4 | 4.88% |
| (N = 82) | | | | | | | | |
| BPH | 0 | 0.00% | 72 | 93.51% | 5 | 6.49% | 5 | 6.49% |
| (N = 77) | | | | | | | | |
| PCA | 2 | 2.38% | 0 | 0.00% | 82 | 97.61% | 2 | 2.38% |
| Stage T1, T2 | | | | | | | | |
| N = 84 | | | | | | | | |
| PCA | 2 | 2.40% | 0 | 0.00% | 81 | 97.59% | 2 | 2.41% |
| Stage T3, T4 | | | | | | | | |
| N = 83 | | | | | | | | |
| Total | | | | | | | 13 | 3.99% |
| Samples | | | | | | | | |
| (N = 326) | | | | | | | | |

B. Test Set

| | | | | |
|---|---|---|---|---|
| Sample | Normal | BPH | PCA | Misclassified Rate |

| | | | | | | | | |
|---|---|---|---|---|---|---|---|---|
| Normal | 15 | 100.00% | 0 | 0.00% | 0 | 0.00% | 0 | 0.00% |
| (N = 15) | | | | | | | | |
| BPH | 0 | 0.00% | 14 | 93.33% | 1 | 6.67% | 1 | 6.67% |
| (N = 15) | | | | | | | | |
| PCA | 3 | 6.67% | 0 | 0.00% | 12 | 80.00% | 3 | 20.00% |
| Stage T1, T2 | | | | | | | | |
| (N = 15) | | | | | | | | |
| PCA | 1 | 6.67% | 1 | 6.67% | 13 | 86.67% | 2 | 13.33% |
| Stage T3, T4 | | | | | | | | |
| (N = 15) | | | | | | | | |
| Total | | | | | | | 6 | 10.00% |
| Samples | | | | | | | | |
| (N = 60) | | | | | | | | |

C. Differentiation of prostate disease from non-disease in the blinded test set

| Disease/Non- | Percent positive (No. Positive/No. Tested) | | | | | |
|---|---|---|---|---|---|---|
| disease | PCA/HM | PCA/BPH | PCA/(BPH/HM) | BPH/HC | BPH/T1, T2 | T1, T2/T3, T4 |
| Sensitivity | 83(25/30) | 83(25/30) | 83(25/30) | 93(14/15) | 93(14/15) | 80(12/15) |
| Specificity | 100(15/15) | 93(14/15) | 97(29/30) | 100(15/15) | 80(12/15) | 87(13/15) |

The classification algorithm correctly predicted 93.51% to 97.59% of the samples for each of the 3 groups in the training set (Table 2A), for an overall correct classification of 96%. The algorithm correctly predicted 90% (54/60) of the test samples with all 15 samples from HM, 93% (14/15) of the BPH samples, and 83% (25/30) of the PCA samples being correctly classified (Table 2B). The sensitive and specificity of the classification system for differentiation disease from the non-disease groups is presented in Table 2C. When comparing PCA vs. non-cancer (BPH/HM), the sensitivity was 83% (25/30) and the specificity was 97% (29/30). The sensitivity of 83% was also obtained when comparing PCA vs. HM (25/30) or PCA vs. BPH (25/30); while the specificity was 100% (15/15) for PCA vs. HM, and 93% (14/15) for PCA vs. BPH. The PPV of the classification system was 96.15% and the NPV was 96.67%.

### Reproducibility

The reproducibility of SELDI spectra, i.e. mass location and intensity, from array to array on a single chip (intra-assay) and between chips (inter-assay) was determined using the pooled normal serum QC sample. Seven proteins in the range of 3000 to 10000 Da observed on spectra randomly selected over the course of the study were used to calculate the coefficient of variance. The intra-assay and inter-assay CV for peak location was 0.05%, and for normalized intensity (peak height or relative concentration) was 15% and 20%, respectively (data not shown). Masses that were within 0.18% mass accuracy between spectra were considered to be the same. Most importantly was the observation that randomly selected samples, blinded to the person performing SELDI and re-run months or even a year later, were correctly classified by the decision tree classification algorithm (FIG. 2C).

Discussion

The current standard screening approach for prostate cancer is a serum test for prostate specific antigen (PSA), and if the test is positive, biopsies are obtained from each lobe of the prostate. Although the PSA test has a sensitivity >90%, its specificity is only 25%. This low specificity results in subjecting men to biopsies of the prostate as well as considerable anxiety when they do not have PCA detectable by biopsy. With the SELDI profiling classification approach, an overall sensitivity of 83%, a specificity of 97%, and a positive predictive value of 96% was obtained in differentiating prostate cancer from BPH and age-matched unaffected healthy men. Provided that this SELDI profiling classification system can be validated using a larger and more clinically diverse study set, this approach would have immediate and substantial benefit in reducing the number of unnecessary biopsies.

Our successful development of a diagnostic system that achieved a high positive predictive value (PPV) (96%) for the blinded test set is based on using a large, carefully chosen training set of randomly selected samples. All specimens were closely age-matched. Serum samples from unaffected HM, identified as having a negative DRE and PSA <4.0 ng/ml, were obtained from the general population during free prostate screening clinics. The majority of the BPH patients had 4-10 ng/ml PSA and multiple negative biopsies, and the PCA patients had cancers ranging from small volume localized disease to local and distant metastatic disease and PSA values varying from 0 to over 8000 ng/ml. Another important factor in the construction of a successful classification system was using an algorithm that could filter out the “noise” that is characteristic of mass spectrometry instruments; the spurious signals created by the EAM and chemical contaminates introduced in the assay; and the natural random daily fluctuations and sample to sample variability. This “normalization” process can be used in, for example, distinguishing peaks due to artifacts from the true peptide/protein peaks. It becomes even more important when considering that most of the protein alterations between cancer and the non-cancer cohorts in the above example is based on either proteins over- or under-expressed and not solely on whether they are present or absent. Accurate and reproducible feature selection or peak “picking” algorithms, with normalization functions, we believe is the an important first step in developing a successful classification algorithm for the SELDI profiling data.

It was encouraging that the three study cohorts could be separated based on the over- or under-expression of nine peptide/protein masses. A previous study from our laboratory (12) is, to the best of our knowledge, the first report describing the concept of SELDI protein profiling as a potential diagnostic approach. This study described that the selection of a combination of multiple proteins resolved by SELDI, dramatically improved the detection rate of early stage bladder cancer compared to a single marker (i.e. urine cytology). Although the differential analysis in this latter study was conducted by cluster analysis and laborious manual visual inspection of all spectra, it did, however, demonstrate the power of SELDI profiling to facilitate the discovery of better cancer biomarkers. Furthermore, it clearly illustrated the need for a bioinformatics algorithm to effectively deal with the high dimensionality of the SELDI data. Based on the results of this previous study, we have explored several different bioinformatics models to mine and analyze the large amounts of data generated from these clinical proteomic studies. The models have included purely biostatistical algorithms, genetic cluster algorithms, support vector machines, and decision classification trees. All have obtained between 83-90% accuracy in separating PCA from the non-cancer (BPH/Normal) samples (G. Wright, O. J. Semmes, P. Barlett, C. Harris, unpublished observations).

The classification tree model was selected because it is easy to interpret and the results can be clearly presented compared to “black box” classifiers such as neural networks and biostatistical algorithms, specifically in regards to the problems associated with the deconvolution steps required in identifying the protein peaks used in the classifiers. With the decision tree algorithm, the protein peaks used in the classifier are easily attainable by examination of the rules, and these rules are easily validated by examination of the SELDI processed spectra. In other studies involving ovarian cancer (17), a discriminator pattern for classification of ovarian cancer consisted of five protein masses of 534 Da, 989 Da, 2111 Da, 2251 Da, and 2465 Da. Although they used a hydrophobic chip chemistry, which might be expected to bind some different proteins then would bind to the IMAC-3Cu chip used in the present study, it is interesting to note that the masses are distinctly different from those used in the prostate classification system. This suggests that the SELDI protein “fingerprint” profiling approach is detecting different protein patterns for each type of cancer. Studies in progress in our laboratory strongly suggest this may be the case. We have observed that SELDI profiles of breast, ovarian, bladder cancer, and leukemia are different from each other and from the prostate classification profile described in this report (G. Wright, Jr., A. Vlahou, C. Laronga, J. Marks, O. J. Semmes, unpublished observations). To assure the robustness of our diagnostic system, the prostate classification algorithm is being challenged with non-prostate cancers and non-prostate diseases to determine that the protein profiling classification algorithm is specific for prostate cancer. A similar scheme will be required of any disease-specific classification system.

One of the goals of this study was to identify markers in the prostate proteome that could potentially be used for early detection of cancer. Ongoing studies in our laboratory evaluating longitudinal serum samples over a 5-10 year period suggested that PCA may be suspected 5 or more years earlier by PSA testing (G. Wright, Jr., P. F. Schellhammer, B-L. Adam, unpublished observations). However, in order to effectively apply this classification system for early detection, it will be important to identify other biomarkers that can distinguish the aggressive, i.e. clinically important, cancers from non-aggressive cancers. Current evidence suggests that preoperative serum PSA below 10 ng/ml is not a useful biomarker for predicting presence, volume, grade, or rate of postoperative failure (4, 18). Thus there is an urgent need for a better biological marker than what PSA and all its molecular forms have been able to provide. A marker proportional to the volume of Gleason grade 4/5 (undifferentiated cancer) represents a critical need to more logically direct therapy tailored to tumor biology. Studies are in progress in our laboratory to evaluate SELDI serum spectra of pre- and post-prostatectomy samples from patients who, after treatment, have biochemical evidence for recurrent disease in an effort to identify the biomarkers or risk factors that signal an aggressive cancer.

The successful use of the prostate classification system described herein relies entirely on the protein “fingerprint” pattern of the nine masses. Since these masses were found to be reproducibly reliably detected, only the mass values are required to make a correct classification or diagnosis. Knowing their identities for the purpose of differential diagnosis is not required. However, because, knowing their exact identities will be essential for understanding what biological role these peptide/proteins may have in the oncogenesis of PCA; potentially leading to novel therapeutic targets. Studies are in progress to purify, identify, and characterize these protein/peptide biomarkers. Furthermore, knowing their identities will make possible production of antibodies for development of either classical or SELDI immunoassays, similar to the single and multiplex formats we previously described for the quantitation of PSA and prostate specific membrane antigen (PSMA) (12, 19). This SELDI immunoassay format provides an alternate platform for quantitation of multiple biomarkers.

The high sensitivity, specificity, PPV and negative predictive value (NPV) obtained by the serum protein profiling approach presented in this example demonstrates that SELDI protein chip mass spectrometry combined with an artificial intelligence classification algorithm can both facilitate discovery (L. H. Cazares, B-L. Adam, M. D. Ward, S. Nasim, P. F. Schellhammer, O. J. Semmes, and G. L. Wright, Jr. Normal, benign, pre-neoplastic, and malignant prostate cells have distinct protein expression profiles resolved by SELDI mass spectrometry, submitted for publication) of better biomarkers for prostate disease and provide an innovative clinical diagnostic platform that has the potential to improve the early detection and differential diagnosis of prostate cancer.

### EXAMPLE 2

### Use of Serum Protein Fingerprinting and a Boosted Decision Tree Analysis for Diagnosing Prostate Cancer or Benign Prostate Hyperplasia

Materials and Methods

Study Groups and Samples

Serum samples were obtained from the Virginia Prostate Center Tissue and Body Fluid Bank. All the samples had been procured from consented patients following protocols approved by Institutional Review Board and stored frozen at −80° C. None of the samples had been thawed more then 2 times. Pre-treatment samples from 99 PCA patients (mean age 71) diagnosed with organ confined cancer, 98 PCA patients (mean age 69) with non-organ confined disease, 92 patients (mean age 67) diagnosed with BPH, and specimens from 96 healthy men (mean age 60) with a negative digitial rectal exam (DRE), a PSA less than 4.0 ng/ml, and no evidence of prostatic disease. The mean PSA values were: healthy men 1.32 ng/ml; BPH 4.60 ng/ml; organ confined PCA 10.10 ng/ml; and non-organ confined 206.93 ng/ml. A quality control sample was prepared by pooling an equal amount of serum from each normal donor, and storing 100 ul aliquots at −80° C.

### SELDI Protein Profiling

Serum samples were prepared by vortexing 20 μl serum with 30 μl of 8M Urea/1% CHAPS in PBS in a 1.5 ml microfuge tube at 4 C for 10 minutes. This was followed by the addition of 100 μl of 1M Urea with 0.125% CHAPS, and the mixture briefly vortexed. Fifty μl of a 1:5 dilution, in PBS, was applied to each well of a bioprocessor (Ciphergen Biosystems, Inc, Fremont, Calif.) containing IMAC-3 chips previously activated with CuSO₄, the bioprocessor sealed and agitated on a platform shaker at a speed of 250 rpm for 30 minutes. A pooled QC serum sample, prepared in the same manner, was applied to an array on each chip used in the study as a reproducibility check. The serum/Urea mixture was discarded and PBS used to wash the chips 3 times, the chips removed from the bioprocessor, washed with DI water (×10), air-dried, and stored in the dark until subjected to SELDI analysis. Just prior to SELDI analysis, 0.5 μl of a saturated solution of sinapinic acid in 50% (v/v) acetonitrile, 0.5% trifluoroacetic acid was applied onto each chip array twice, letting the array surface air dry between each application. Chips were placed in the PBS-II mass spectrometer (Ciphergen Biosystems, Inc.), and time-of-flight spectra generated by averaging 192 laser shots (positive mode, laser intensity 220, detector sensitivity 7, and focus lag time of 900 ns). Mass accuracy was calibrated externally using the All-in-1 peptide MW standard (Ciphergen). Peak detection and alignment was performed using Ciphergen ProteinChip Software 3.0 with slight modifications. The mass range from 2000 to 40000 Da was selected for analysis because this range contained the majority of the resolved protein/peptides.

Data Analysis

### Feature Selection

The power of each peak in discriminating PCA from normal, BPH from normal, and BPH from PCA was determined by estimating the AUC. The area under the ROC curve ranges from 0.5 (no discrimination) to 1.0 (absolute prediction) (6). Peaks with and AUC below 0.62 were excluded from further data analyses.

Boosted Decision Stump Classifier

The classifier was developed using a training data set consisting of 167 PCA, 77 BPH, and 82 normal serum samples. The validity and accuracy of the classification algorithm was then challenged with a blinded test data set consisting of 30 PCA, 15 BPH, and 15 normal samples.

The AdaBoost algorithm described by Freund and Schapire (7-9), was used with modifications to construct the classifier. In this algorithm, decision ‘stumps’ are used as the base classifiers, each of which has only one split, using one peak. A decision stump usually is a weak classifier, with rather high error rate. However, the combined stumps using weighted vote is expected to be a very accurate classifier. The decision stump is denoted by (Z, c) where Z is a peak, selected from the peaks in the training set, and c is a threshold. The Ah training sample is denoted by (y\dot{r}; Zi1, . . . ; Zip), where yi stands for the class (label) of observation i (i=1; . . . , N) and Zij is the intensity of the ith peak of Ah observation. If there are two classes, class one (e.g. non-cancer) is denoted by yi=1, and class two (e.g. cancer), is denoted by yi=−1. The stump has two leaves, the left one contains the training samples with intensity of peak Z less than or equal to the threshold c, and the right leaf contains all other samples. If most of the samples in the left leaf are, for example cancer, then the samples with Z≦c will be classified as cancer. The classifier is denoted as f(x), where x is a Boolean variable, “Z≦c”, and f(x) takes value from {−1, 1}: f(xi)=1 if the ith training sample is classified as class one; and f(xi)=−1 if the ith sample is classified as class two. The sample is misclassified if y\dot{i}(xi)=−1. For the left leaf, Z≦c, or x=“true”, let n₁₁ and n₂₁ be the numbers of observations with yi=1 and yi=−1, respectively, i.e.,

$$n 11 = {\sum }_{i = 1}^{N} I \{ ( y i = 1 ) \& ( Z \leq c ) \} , n 21 = {\sum }_{i = 1}^{N} I \{ ( yi = - 1 ) \& ( Z \leq c ) \} , \qquad ( 1 )$$

where i (statement) is the indicator function, which equals 1 if the statement is true, or 0 if the statement is false. Similarly, let n₁₂ and n₂₂ be the numbers of observations for yi=1 and yi=−1, respectively, and Z>c, i.e.,

$$n 12 = {\sum }_{i = 1}^{N} I \{ ( yi = 1 ) \& ( Z > c ) \} , n 22 = {\sum }_{i = 1}^{N} I \{ ( yi = - 1 ) \& ( Z > c ) \} . \qquad ( 2 )$$

Then the log likelihood for this multinomial model is

$$log L = {\sum }_{u = 1}^{2} {\sum }_{v = 1}^{2} {n}_{uv} log ( {p}_{uv} ) , \qquad ( 3 )$$

where pᵤᵥ is evaluated by nᵤᵥ=(n1v+n2v). The peak Z and its threshold c are obtained by maximizing the log likelihood. The following threshold values were utilized in this example for the prostate cancer biomakers (molecular weight of protein biomarker in parenthesis): 0.1912 (9656); 1.0519 (9720); 0.0000 (6542); 0.0000 (6797); 2.2427 (6949); 0.0000 (7024); 0.1638 (8067); 1.7755 (8356); 13.8103 (3963); 0.8301 (4079); 0.3805 (7885); and 0.0000 (6990). The following threshold values were utilized in this example for the benign prostate hyperplasia biomarkers (molecular weight of protein biomarker in parenthesis: 0.0000 (for 7820, 4580, 7844, 4071 and 6099); 0.2679 (7054); 0.1991 (5298); 3.3758 (3486); and 20.1535 (8943). Through these threshold values, the continuous activities are converted to binary (or logic) values.

In developing the boosting classifier, an ensemble of decision stumps on weighted observations is created. If for example, weight wᵢ is assigned to the ith observation yᵢ so that the sum of the weights is N. Then equation (3) is still used to find the split (Z, c), except that the counts n₁₁; n₂₁; n₁₂ and n₂₂ should be modified to incorporate the weights. Equation (1) becomes

$${n}_{11} = {\sum }_{i = 1}^{N} {w}_{i} I \{ ( yi = 1 ) \& ( Z \leq c ) \} , {n}_{21} = {\sum }_{i = 1}^{N} {w}_{i} I \{ ( {y}_{i} = - 1 ) \& ( Z \leq c ) \} ,$$

and equation (2) becomes

$${n}_{11} = {\sum }_{i = 1}^{N} {w}_{i} I \{ ( {y}_{i} = 1 ) \& ( Z > c ) \} , {n}_{22} = {\sum }_{i = 1}^{N} {w}_{i} I \{ ( {y}_{i} = - 1 ) \& ( Z > c ) \} .$$

Thus the basic concept of boosting algorithm is to construct an ensemble of base classifiers on weighted observations. For the first round, equal weights to all observations are used, i.e., wᵢ=1 for i=1, . . . , N. The samples are classified, and for the next round the weights are increased for the misclassified observations in the previous round, while decreasing the weights for the correctly classified observations. Therefore the next decision stump focuses on the 4 samples misclassified by the previous stump. This procedure is repeated again and again, as outlined below, until a defined number of stumps have been created.
1. For the first round, using equal weights, wᵢ=1 (i=1, . . . , N).
2. For m=1 to M: Construct a decision stump fₘ(x) for the training data with weights wᵢᵥ² Compute error rate

$${err}_{m} \frac{1}{N} {\sum }_{i = 1}^{N} {w}_{i} I ( {y}_{i} {f}_{m} ( {x}_{i} ) = - 1$$
3. Compute confidence αₘ=log {(1−errₘ)/errₘ}.
4. Update weights, set wᵢ←wᵢ. exp {αₘ.I(yᵢfₘ(xi)=1)}, i=1, . . . N,
and normalize the weights: Σᵢwi=N.
5. Use the linear combination

$$f ( x ) = {\sum }_{m = 1}^{M} {α}_{m} {f}_{m} ( x )$$

as the final combined classifier.

The combined classifier f(x) is a weighted majority vote of the M base classifiers. For the ith sample, the mth base classifier fₘ(xᵢ)=1 if it is classified as class one, and fm(xi)=−1 if it is classified as class two. The contribution of the mth decision stump to the final vote is either αm, if the votes for class 1, or −αm, if the votes for class 2. Therefore, if the total vote is positive, i.e.,

$$f ( {x}_{i} ) = {\sum }_{m = 1}^{M} {α}_{m} {f}_{m} ( {x}_{i} ) > 0 , i = 1 , … , N , \qquad ( 4 )$$

and the majority votes for class 1, then the sample is classified as class 1; and it is classied as class 2, if (f(xi)≦0). This is a weighted majority vote because am are not equal.

Because there are so many variables involved in a classifier, the testing or generalization error is zero, with no evidence of over-fitting. A detailed explanation why boosting methods can avoid over-fitting has been described by Schapire and Freund (10). A sample in the training set was classified correctly if and only if yif(xi)>0. The latter is composed of two parts:

$${y}_{i} f ( {x}_{i} ) = {\sum }_{m = 1}^{M} {α}_{m} {y}_{i} {f}_{m} ( x ) = {\sum }_{m = 1}^{M} {α}_{m} I ( {y}_{i} {f}_{m} ( x ) > 0 ) - {\sum }_{m = 1}^{M} {α}_{m} I ( {y}_{i} {f}_{m} ( x ) \leq 0 ) , i = 1 , … , N ,$$

where the weights am have been normalized (Σm αm=1). The first item in the right side of the equation is the total vote of the voters (M) who made the correct decisions (since yᵢfₘ(x)>0), and the second item is the total vote of the voters who made the wrong decisions. yᵢf(xᵢ) is referred to as the margin of the ith sample. A sample with a negative margin has been misclassified by the combined classifier. The proportion in the training set with negative margins is the training error rate. The minimal value of the margins is determined by:

$$minimal {margin}_{M} = {min}_{1 < i \leq N} \{ {\sum }_{m = 1}^{M} {α}_{m} {y}_{i} {f}_{m} ( x ) \}$$

and is an important quantity for voting methods. If the minimal margin is above zero, there will be no training samples being misclassified. The larger the minimal margin, the less chance of a sample being misclassified (i.e., higher confidence in generalization). Therefore, the minimal margin in the training samples measures how well the two classes are separated apart by the learning algorithm in both the training and test sets. As the minimal margin keeps increasing, there is larger and larger room for the test samples to be correctly classified by the combined classifier. The plot of minimal margins is important in deciding when to stop adding more base classifiers (11).

### Statistical Analyses

The AUC of the Receiver Operating Characteristics (ROC) was computed to identify the peaks having the highest potential to discriminate the 3 groups (6). Specificity was calculated as the ratio of the number of non-disease samples correctly classified to the total number of non-disease samples. Sensitivity was calculated at the ration of the number of correctly classified diseased samples to the total number of diseased samples.

Results

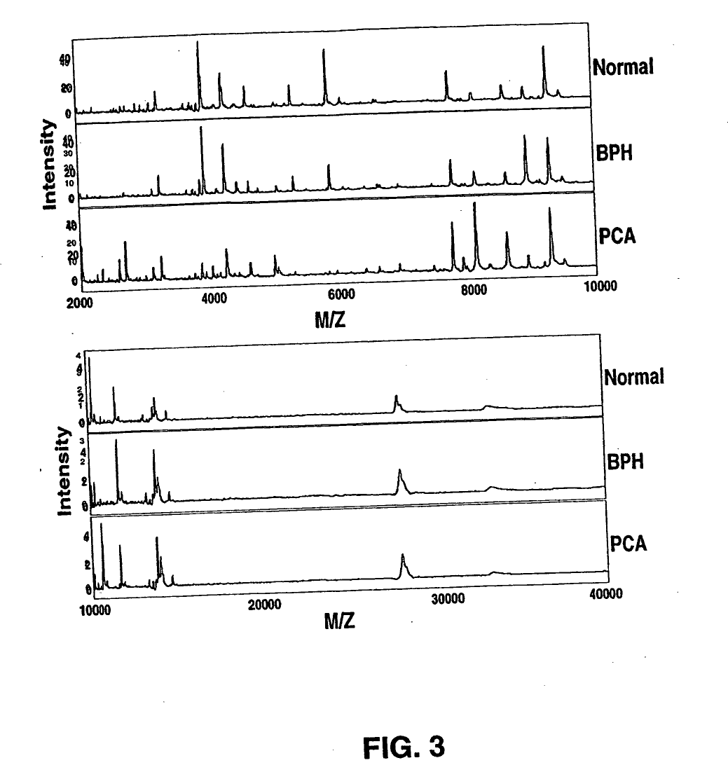

Each SELDI spectrum revealed an average of 80 peak masses in the 2000-40000 Da range. The QC spectra were found to be very reproducible with an intra- and inter-assay CV for peak location of 0.05%, and a CV of 15% and 20%, respectively for peak intensity (data not shown). FIG. 3 shows representative examples of the SELDI spectra. Analysis of all 772 spectra (336 samples run in duplicate) identified 779 peaks, of which 124 had an AUC equal to or greater than 0.62. These 124 peaks identified in the training set were used to construct the classifier.

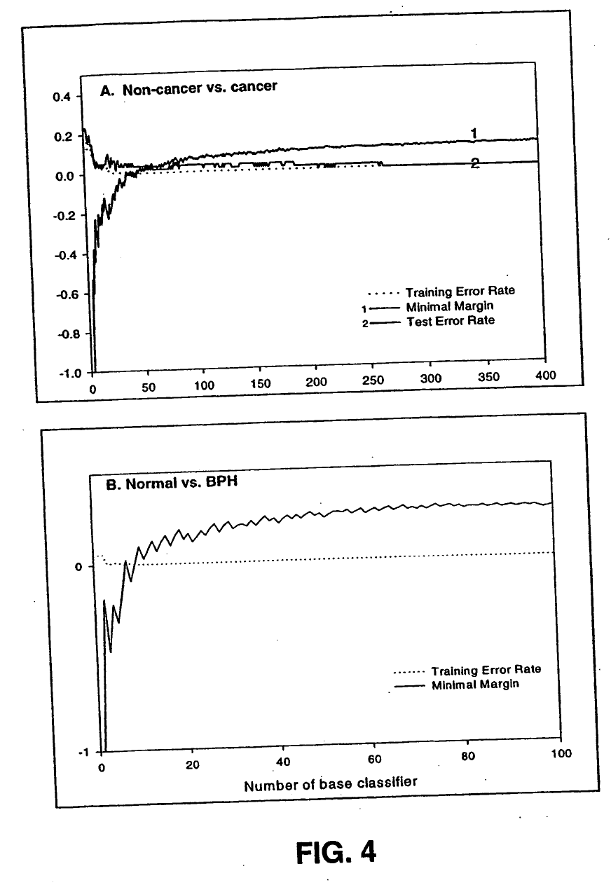

One of the concerns in construction and use of learning algorithms is the possibility of over-fitting the data. However, boosting methods can avoid over-fitting by calculating the minimal margin. The larger the minimal margin the less chance of a test sample being misclassified. FIG. 4A shows the training error rate, the minimal margin, and the generalization error rate (testing error) against M, the number of base stumps for the boosted decision tree classifier distinguishing non-cancer from cancer. After the training error reaches zero (round 47), the minimal margin keeps increasing, and at the same time, the generalization error keeps decreasing, finally reaching zero on round 265, and then stays at zero. Similarly, FIG. 4B shows the training error rate and the minimal margin against the number of base stumps for the boosted decision tree classifier distinguishing normal from BPH. Again, after the training error reaches zero (on round 9), the minimal margin keeps increasing. As shown, there is no evidence of over-fitting for the boosting technique. The learning process does not stop when the training error becomes zero; on the contrary, the learning algorithm continues to enlarge the minimal margin between the two classes. Therefore, as long as the minimal margin keeps increasing, adding more base classifiers will not cause over-fitting.

The first boosting classifier (AdaBoost Classifier) for distinguishing non-cancer from PCA, consisted of 400 base classifiers, including 62 peaks, with a 0 error rate in both 326 training samples and in 60 testing samples. When the number of base stumps (i.e., the number of rounds) was greater than 47, the training error was zero, but the testing error (generalization error), was 0.0333. The generalization error was found to decline slowly as the number of base stumps increases. After round 265, the generalization error remained zero. The 100 decision stumps with 12 peaks for distinguishing normal from BPH also obtained a 0 error rate for both the 159 training and 30 test samples. In this case, the training error became zero on round 9, and the generalization error for 30 test samples was s 0, beginning with round 1. When combining these two boosted decision stumps, 100 percent separation for the three classes: normal, BPH and PCA was achieved (Table 1).

However, this classifier combined 500 base classifiers using 74 peaks. For the purpose of interpretation, there is a need to know what peaks are most important in distinguishing cancer from noncancer, and what peaks are most important in distinguishing BPH from normal. This is a problem of feature selection in machine learning. There are many methods for feature selection, which is an intrinsic component in decision tree models. In fact, using the areas under the ROC curves to select 124 from 779 peaks is the first step of feature selection, called the filter method. In constructing the boosted decision stump classifier, one peak is selected from the 124 peaks in each round. Because this feature selection procedure is embedded in the algorithm, a feature (peak) may be selected many times. To avoid selecting the same peak several times, and to select only a new peak in each round, the features previously selected are ignored. This algorithm is referred to as Boosted Decision Stump Feature Selection (BDSFS) (12). Using this method, the identity of the protein masses having utility in distinguishing PCA from BPH and normal men can readily be obtained. Table 3 lists the average molecular weights for the first 12 peaks selected for distinguishing cancer from non-cancer by using the BDSFS algorithm, and the first 9 peaks selected for distinguishing between BPH from normal. Table 4 presents the results of the BDSFS algorithm. These tables are set forth below:

TABLE 3
Peak masses used by the Boosted Decision Stump
Feature Selection classifier in the order of their selection.

| | |
|---|---|
| Non-cancer | Normal |
| vs. Cancer | vs. BPH |

| No. | Mass | No. | Mass |
|---|---|---|---|
| 1 | 9655.75 | 1 | 7819.75 |
| 2 | 9719.99 | 2 | 4579.73 |
| 3 | 6541.82 | 3 | 7844.00 |
| 4 | 6797.02 | 4 | 4071.18 |
| 5 | 6949.22 | 5 | 7054.17 |
| 6 | 7024.02 | 6 | 5297.55 |
| 7 | 8066.95 | 7 | 3486.21 |
| 8 | 8355.56 | 8 | 6099.08 |
| 9 | 3963.18 | 9 | 8943.08 |
| 10 | 4079.48 | | |
| 11 | 7884.72 | | |
| 12 | 6990.63 | | |

TABLE 4
Classification of the training and test sets by the Boosted
Decision Stump Feature Selection algorithm.

| | | Training Set | | | Test Set | | |
|---|---|---|---|---|---|---|---|
| Prediction | | Normal | BPH | PCA | Normal | BPH | PCA |
| BDSFS | Normal | 82 | 0 | 7 | 14 | 0 | 0 |
| Classifier | BPH | 0 | 74 | 0 | 0 | 15 | 1 |
| | PCA | 0 | 3 | 160 | 1 | 0 | 29 |

| | | |
|---|---|---|
| Sensitivity | 95.18% | 96.67% |
| Specificity | 98.11% | 96.67% |

| | |
|---|---|
| # of Peaks | 21 peaks from 21 base classifiers |
| Minimal | −0.2555 |
| Margin | |

This classifier (BDSFS Classifier) used 21 peaks selected by the BDSFS algorithm, which consisted of the 12 peaks in Table 3 for distinguishing cancer from non-cancer, and the first 9 peaks for distinguishing normal from BPH. This classifier obtained a sensitivity and specificity in the test set of 96.67%. In this case, the interpretation is much easier than the AdaBoost Classifier, which contains 74 peaks (Table 1). However, the minimal margin for the BDSFS classifier is −0.2555, while the minimal margin for classifier 1 is 0.1143. Therefore, the AdaBoost Classifier will be more accurate than the BDSFS Classifier for new (unknown) samples.

### Discussion

SELDI mass spectrometry, using a protein chip which captures proteins based on their ability to selectively bind to chemically activated copper surface through histidine, tryptophan, cysteine, or phosphorylated amino acids, was capable of resolving an average of 80 serum protein/peptides, ranging from 2,000-40,000 Daltons. This is far less then the hundred to thousands of proteins capable of being separated by two-dimensional electrophoresis; however the advantage over 2D-EP is the ability of SELDI to effectively resolve polypeptides and peptides under 20,000 Da. This has opened the door to readily resolve and study such peptides as potential biomarkers for diagnosis, prognosis, and as therapeutic targets. Interestingly, the 24 proteins identified in this study to be the most useful for separating PCA from the non-cancer groups, range between 3,000-10,000 Da. Since the introduction of SELDI, there has been a concern that such “peptides” might represent random fragments of larger proteins. If this were true, we would not have been able to achieve high reproducibility in protein patterns (0.05% CV for peak location and 15-20% CV for peak intensities).

Even with only an average of 80 peaks per spectra obtained between 2-40 KDa, there is still extremely high dimensionality of the data. Initial analysis of the 772 serum samples (386 run in duplicate) resulted in 63,157 peaks, which were reduced to 779 peaks after cluster analysis and peak alignment. Of the 779 peaks, 124 peaks were statistically found to have the highest potential to discriminate the 3 groups: normal vs. PCA, normal vs. BPH; and BPH vs. PCA. Subsequent analysis of the 124 peaks in each of the 772 samples resulted in processing over 95,000 data points to order to identify the pattern or combination of masses that separate PCA from the non-cancer groups. Because of this high dimensionality, only an artificial intelligent algorithm would be capable of analyzing such high volume of data to develop an efficient and reproducible classifier. We have evaluated several different models, including biostatistical (13,14), genetic cluster, and support vector machines algorithms. Although most could obtain between 83-90% accuracy in differentiating PCA from the non-cancer (BPH/normal) groups, the decision tree model (15) was selected because it is easier to interpret compared to “black box” classifiers such as neural networks and biostatistical algorithms. Using the same data set described in this study, we developed a single decision tree base classifier with nine masses between 2000-10000 Da that achieved a sensitivity of 83% and a specificity of 97% for differentiating PCA from the non-cancer groups (i.e., BPH and Normal) (discussed in Example 1). However, a single decision tree classifier's predictive power may not be as good as other learning algorithms, such as neural networks and surface vector machine. Furthermore, assays for the early detection of cancer need to be highly accurate to avoid generating too many false positives. The present study was initiated to determine if we could increase the predictive power of the decision tree classifier. Tremendous improvement in the predictive power of decision tree classifiers has been recently reported using voting methods, such as boosting (16) and bootstrap methods. In one voting method, called the bagging method (17,18), the decision tree model is fitted many times on randomly re-sampled observations (bootstrap sub-samples) and then combines the decision trees using simple voting. Another approach is a boosting method (7), referred to as the AdaBoost algorithm, which fits the decision tree model many times on weighted observations, and then combines the decision trees using weighted voting. In both bagging and boosting, the combined classifier has better performance than each of the individual base decision trees. We chose the boosting approach over the bagging algorithm because it is generally more accurate in the test samples than the bagging approach (10). Using the AdaBoost algorithm a classifier was established that was 100% accurate in predicting, for both the training and blinded test sets, whether the sample was from a patient diagnosed with PCA or BPH, or if the sample was from a healthy donor. Although this classifier produced a sensitivity and specificity of 100%, it used 74 protein mass values (peaks), and required combining 500 base decision tree classifiers, making it highly accurate but difficult to interpret. Other models, such as a biostatistical approach using Wavelets (13) and surface vector machines (Wright, unpublished data), can reach similar high accuracy but with the same difficulty, especially in identifying the protein masses used in the classifier. This difficulty results when the same feature (peak) is selected many times. To avoid selecting the same peak several times, we used a modified boosting algorithm called the Boosted Decision Stump Feature Selection (12), which selects only a single peak on each round, and excludes peaks selected from previous rounds. In this way, the identity of the 24 peaks important in distinguishing the three groups was easily obtained. The classifier was slightly less accurate than the AdaBoost classifier by misclassifying 1 of 15 BPH as PCA and 1/29 PCA as normal; whereas all 14 samples from normal unaffected men were correctly identified. This classifier still achieved a respectable 96.67% for both sensitivity and specificity, using 21 peaks and only 21 base decision tree classifiers. The specificity remained the same at 97% as obtained with the single base classifier but the sensitivity, the ability to correctly predict the PCA samples, was increased from 83% to 97% by this boosting algorithm.

The PSA test is the current screening test for prostate cancer, and if positive, biopsies are obtained from each lobe of the prostate. Many consider this test the best for any human cancer, yet it is far from a perfect test for early detection of PCA. Although it has a high sensitivity of >90%, its specificity is only 25% in distinguishing PCA from BPH; and some men with prostate cancer have normal levels of PSA. Because of the low specificity, men are subjected to unnecessary biopsies causing considerable anxiety when they in fact do not have cancer. Current evidence also suggests that preoperative serum PSA below 10 ng/ml is not a useful biomarker for predicting presence, volume, grade, or rate of postoperative failure (1). Based on these facts there is a need for better biological marker than PSA and all its molecular forms can provide. Provided that the accuracy of the boosting decision tree classifier can be validated on a larger number of samples, evaluated at multiple sites, including testing the validity of the profiling assay with samples from non-cancer patients, SELDI protein profiling combined with a bioinformatics classifier may provide that “better” test for the early detection and diagnosis of prostate cancer. Support for this potential are reports from other investigators achieving similar results for ovarian and breast cancer using SELDI combined with different bioinformatics classifiers then used in the present study (19; Li, J, Zhang, Z, Rosenzweig, J, Wang, Y Y, Chan, D. Potential serum biomarkers identified by SELDI mass spectrometry can discriminate breast cancer from non-cancer patients. Proc. Am. Assoc. Cancer Res. 2002; 43:136 [abstract 682]). Overall these initial studies suggest that SELDI provides a unique opportunity to develop an innovative proteomic approach to cancer diagnosis.

The identity of the peak masses used in the classifier is not necessary for making a diagnosis. The only requirement for this classification system to make an accurate diagnosis is that the biomarkers be reproducibly detected by SELDI and selected by the classifier. Obtaining a name for each of the masses used in the classifier will not make the classification system better or more accurate. Knowing the identity of the protein biomarkers is, however, important from a discovery perspective. The identity of the peptide/protein biomarkers will be needed to understand the biological role these proteins have in the oncogenesis of PCA. Such information could lead to better therapeutic interventions. Knowing the identity will facilitate the production of antigen and antibody reagents for development of classical multiplex immunoassays and antibody arrays, should the profiling approach fail to be developed into a clinical assay. For these reasons, protein identification of the potential biomarkers is in progress.

In conclusion, the high sensitivity and specificity achieved by the combined use of multiple serum biomarkers provides supporting evidence that SELDI, combined with a learning algorithm, can not only facilitate the discovery of new and better biomarkers for PCA, but has potential for being developed into a novel clinical diagnostic assay.

### REFERENCES CITED IN EXAMPLE 1

1. Howe, H. L., Wingo, P. A., Thun, M. J., Ries, L. A., Rosenberg, H. M., Feigal, E. G., and Edwards, B. K. Annual report to the nation on the status of cancer (1973 through 1998), featuring cancers with recent increasing trends. J. Natl. Cancer Inst., 93: 824-842, 2001.
2. Djavan, B., Zlotta, A., Kratzik, C., Remzi, M., Seitz, C., Schulman, C. C., and Marberger, M. PSA, PSA density, PSA density of transition zone, free/total PSA ratio, and PSA velocity for early detection of prostate cancer in men with serum PSA 2.5 to 4.0 ng/mL. Urology, 54: 517-522, 1999.
3. Pannek, J. and Partin, A. W. The role of PSA and percent free PSA for staging and prognosis prediction in clinically localized prostate cancer. Semin. Urol. Oncol., 16: 100-105, 1998.
4. Stamey, T. A., Johnstone, I. M., McNeal, J. E., Lu, A. Y., and Yemoto, C. M. Preoperative serum prostate specific antigen levels between 2 and 22 ng/ml correlate poorly with post-radical prostatectomy cancer morphology: Prostate specific antigen cure rates appear constant between 2 and 9 ng/ml. J. Urol. 167:103-111, 2002.
5. Srinivas, P. R., Srivastava, S., Hanash, S., and Wright, Jr., G. L. Proteomics in early detection of cancer. Clin. Chem. 47:1901-1911, 2001.
6. Adam, B-L., Vlahou, A., Semmes, O. J., and Wright, Jr., G. L. Proteomic approaches to biomarker discovery in prostate and bladder cancers. Proteomics 1: 1264-1270, 2001.
7. Chong, B. E., Hamler, R. L., Lubman, D. M., Ethier, S. P., Rosenspire, A. J., and Miller, F. R. Differential screening and mass mapping of proteins from premalignant and cancer cell lines using nonporous reversed-phase HPLC coupled with mass spectrometric analysis. Anal. Chem., 73: 1219-1227, 2001.
8. Ferrari, L., Seraglia, R., Rossi, C. R., Bertazzo, A., Lise, M., Allegri, G., and Traldi, P. Protein profiles in sera of patients with malignant cutaneous melanoma. Rapid Commun. Mass Spectrom., 14:1149-1154, 2000.
9. Keough, T., Lacey, M. P., Fieno, A. M., Grant, R. A., Sun, Y., Bauer, M. D., and Begley, K. B. Tandem mass spectrometry methods for definitive protein identification in proteomics research. Electrophoresis, 21: 2252-2265, 2000.
10. Merchant, M. and Weinberger, S. R. Recent advancements in surface-enhanced laser desorption/ionization-time of flight-mass spectrometry. Electrophoresis, 21: 1164-1177, 2000.
11. Kuwata, H., Yip, T. T., Yip, C. L., Tomita, M., and Hutchens, T. W. Bactericidal domain of lactoferrin: detection, quantitation, and characterization of lactoferricin in serum by SELDI affinity mass spectrometry. Biochem. Biophys. Res. Commun., 245: 764-773, 1998.
12. Wright, G. L., Jr., Cazares, L H, Leung, Sau-Mei, Nasim, S, Adam, B L, Yip, T T, Schellhammer, P F, and Vlahou, A. Proteinchip Surface Enhanced Laser Deorption/ionization (SELDI) Mass Spectromotry: A Novel Proteomic Technology fro Detection of Prostate Cancer Biomarkers in Complex Protein Mixtures. Prostate Cancer and Prostate Diseases 2, 264-267.1-1-1999.
13. Vlahou, A., Schellhammer, P. F., Mendrinos, S., Patel, K., Kondylis, F. I., Gong, L., Nasim, S., and Wright, J. G., Jr. Development of a novel proteomic approach for the detection of transitional cell carcinoma of the bladder in urine. Am. J. Pathol., 158: 1491-1502, 2001.
14. Breiman, L., Friedman, J. H., Olshen, R. A., and Stone, C. J. Classification and Regression Trees, Wadsworth International Group, Belmont, Calif. (1984), pp. 103-111 and 203-215.
15. Pepe, M. S., Receiver operating characteristic methodology, Amer. Statistical Assoc., 95:308-311, 2000.
16. Gelman, A., Carlin, J. B., Stern, H. S., and Rubin, D. B. Bayesian Data Analysis. Chapman & Hall, Londan (1995).
17. Stamey, T. A. Preoperative serum prostate specific antigen (PSA) below 10 ug/L predicts neither the presence of prostate cancer nor the rate of postoperative PSA failure. Clin. Chem. 47:631-634, 2001.
18. Petricoin, III, E. F., Ardkani, A. M., Hitt, B. A., Levine, P. J., Fusaro, V. A., Steinberg, S. M., Mills, G. B., Simone, C., Fishman, D. A., Kohn, E. C., Liotta, L. A. Use of proteomic patterns in serum to identify ovarian cancer. The Lancet 359:572-577, 2002.
19. Xiao, Z., Adam, B-L., Cazares, L. H., Clements, M. A., Davis, J. W., Schellhammer, P. F., Dalmasso, E. A., Wright, Jr., G. L. Quantitation of serum prostate-specific membrane antigen by a novel protein biochip immunoassay discriminates benign from malignant prostate disease. Cancer Res. 61:6029-6033, 2001.

### REFERENCES CITED IN EXAMPLE 2

1. Stamey, T A, Johnstone, I M, McNeal, J E, Lu, A Y, Yemoto, C M. Preoperative serum prostate specific antigen levels between 2 and 22 ng/ml correlate poorly with post-radical prostatectomy cncer morphology: Prostate specific antigen cure rates appear constant between 2 and 9 ng/ml. J Urol 2002; 167:103-111.
2. Merchant, M, Weinberger, S R. Recent advancements in surface-enhanced laser desorption/ionization-time of flight-mass spectrometry. Electrophoresis 2000; 21:1164-1177.
3. Kuwata, H, Yip, T T, Yip, C L, Tomita, M, Hutchens, T W. Bactericidal domain of lactoferrin: detection, quantitation and characterization of lactoferricin in serum by SELDI affinity mass spectrometry. Biochem Biophys Res Commun 1998; 245:764-773.
4. Wright, Jr, G L, Cazares, L H, Leung, S-M, Nasim, S, Adam, B L, Yip, T T, et al. Proteinchip surface enhanced laser desorption/ionization (SELDI mass spectrometry: A novel proteomic technology for detection of prostate cancer biomarkers in complex protein mictures. Prostate Cancer & Prostate Diseases 1999; 2:264-267.
5. Vlahou, A, Schellhammer, P F., Mendrinos, S, Patel, K, Kondylis, F I, Gong, L, et. al. Development of a novel proteomic approach for the detection of transitional cell carcinoma of the bladder in urine. Am J Pathol 2001; 158:1491-1502.
6. Pepe, M S. Receiver operating characteristic methodology. Amer Statistical Assoc. 2000; 95: 308-311.
7. Freund, Y, Schapire, R. A decision-theoretical generalization of on-line learning and an application to boosting. J Computer & Syst Sci. 1997; 55:119-139.
8. Hastie, T, Tibshirani, R, Friedman, J. The Elements of Statistical Learning. New York: Springer-Verlag. 2001; 301 pp.
9. Friedman, J, Hastie, T, Tibshirani, R. Additive logistic regression: A statistical view of boosting. The Annals of Statistics. 2000; 28: 337-407.
10. Schapire, R, Freund, Y. Boosting the margin: A new explanation for the electiveness of voting methods. The Annals of Statistics/1998; 26: 1651-1686.
11. Vapnik, V N. The Nature of Statistical Learning Theory. New York: Springer-Verlag. 1995.
12. Das, S. Filters, wrappers and a boosting-based hybrid for feature selection. In Proceeding of the Eighteenth International Conferences on Machine Learning. San Francisco, Calif.: Morgan Kaufmann Publishers, 2001; 74-81 pp.
13. Qu, Y, Adam, B-L, Thornquist, M, Potter, J, Yasui, Y, Davis, J, et al. Data reduction using discrete wavelet transform in discriminant analysis with very high dimension. 2002; Biometrics (In Press).
14. Yasui, Y. Pepe, M, Thompson, M L, Adam, B L, Wright, G. Jr, Qu, Y, et al. A data-analytic strategy for protein-biomarker discovery: Profiling of high-dimensional proteomic data for cancer detection. 2001; University of Washington, Department of Biostatistics, Technical Report No 177.
15. Breiman, L, Friedman, J H, Olsen, R A, Stone, C. J. Classification and Regression Trees. Belmont, Calif.: Wadsworth International Group, 1984.
16. Drucker, H, Cortes, C. Boosting decision trees. In Advances in Neural Information Processing Systems 1996; 8: pp 479-485.
17. Breiman, L. Bagging predictors. Machine Learning Breiman, L. 1996. 24:123-140.
18. Breiman, L. Statistical modeling: The two cultures. Statistical Science. 2001; 16: 199-231.
19. Petricoin, III, E F, Ardkani, A M, Hitt, B A, Levine, P J, Fusaro, V A, Steinberg, S M, et al. Use of proteomic patterns in serum to identify ovarian cancer. The Lancet 2002; 359:572-577.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method for aiding in a diagnosis of benign prostate hyperplasia or prostate cancer, comprising:
(a) detecting at least two protein biomarkers in a test sample from a subject, said protein biomarkers having a molecular weight selected from the group consisting of about 4475±81, about 5074±91, about 5382±97, 7024±13, about 7820±14, about 8141±15, about 9149±16, about 9508±17, and about 9656±17 Daltons; and
(b) correlating the detection with a probable diagnosis of benign prostate hyperplasia, prostate cancer or a negative diagnosis.

2. The method of claim 1, wherein said detection step further comprises identifying the differential expression of said biomarkers.

3. The method of claim 1, wherein the at least two protein biomarkers are the about 7820±14 Dalton and the 7024±13 Dalton protein biomarkers.

4. The method of claim 1, wherein the at least two protein biomarkers are the about 7820±14 Dalton, the about 7024±13 Dalton, the about 5382±97 Dalton, and the about 4475±81 Dalton biomarkers.

5. The method of claim 1, wherein the at least two protein biomarkers are the about 8141±15 Dalton, about 9149±16 Dalton, and the about 9656±17 Dalton biomarkers.

6. The method of claim 1, wherein the at least two protein biomarkers are the about 5074±91 Dalton, the about 9149±16 Dalton, and the about 9656±17 Dalton biomarkers.

7. The method of claim 1, wherein the at least two protein biomarkers are the about 9149±16 Dalton and the about 9508±17 Dalton biomarkers.

8. The method of claim 1, wherein the at least two protein biomarkers are the about 5382±97 Dalton, the about 7024±13 Dalton, and the about 7820±14 Dalton biomarkers.

9. The method of claim 1, wherein the at least two protein biomarkers are the about 7024±13 Dalton, the about 7820±14 Dalton, the about 5382±97 Dalton, and the about 4475±81 Dalton biomarkers.

10. The method of claim 3, wherein said method comprises determining the absence of the about 7820±14 Dalton protein biomarker and determining the presence of the about 7024±13 Dalton protein biomarker.

11. The method of claim 4, wherein said method comprises determining the absence of the about 7820±14 Dalton and about 7024±13 Dalton biomarkers, the presence of the about 5382±97 Dalton biomarker, and the quantity of the about 4475±81 Dalton biomarker.

12. The method of claim 5, wherein said method comprises determining the quantity of the about 8141±15 Dalton, about 9149±16 Dalton, and about 9656±17 Dalton biomarkers.

13. The method of claim 6, wherein said method comprises determining the quantity of the about 5074±91 Dalton, about 9149±16 Dalton, and about 9656±17 Dalton biomarkers.

14. The method of claim 7, wherein said method comprises determining the quantity of the about 9149±16 Dalton and about 9508±17 Dalton biomarkers.

15. The method of claim 8, wherein said method comprises determining the absence of the about 5382±97 Dalton, about 7024±13 Dalton and about 7820±14 Dalton biomarkers.

16. The method of claim 9, wherein said method comprises determining the absence of the about 7024±13 Dalton and about 7820±14 Dalton biomarkers, the presence of the about 5382±97 Dalton biomarker and determining the quantity of the about 4475±81 Dalton biomarker.

17. The method of claim 1, wherein said detecting at least two protein biomarkers in a test sample from a subject is performed by mass spectroscopy.

18. The method of claim 17, wherein said mass spectroscopy is laser desorption mass spectroscopy.

19. The method of claim 18, wherein said mass spectroscopy is surface enhanced laser desorption/ionization mass spectroscopy.

20. The method of claim 19, wherein the laser desorption/ionization mass spectroscopy includes:
(a) providing a substrate comprising an adsorbent attached thereto;
(b) contacting the test sample with the adsorbent;
(c) desorbing and ionizing the biomarkers from the substrate; and
(d) detecting the desorbed/ionized biomarkers with a mass spectrometer.

21. The method of claim 20, further comprising purifying the test sample prior to contacting the test sample with the adsorbent.

22. The method of claim 1, wherein said detecting at least two protein biomarkers in a test sample from a subject is performed by an immunoassay.

23. The method of claim 22, wherein said immunoassay is an enzyme immunoassay.

24. The method of claim 1, wherein said test sample is a biological fluid from said subject.

25. The method of claim 24, wherein the biological fluid is blood serum.

26. The method of claim 1, wherein the test sample is selected from the group consisting of seminal fluid, seminal plasma, saliva, blood, lymph fluid, lung/bronchial washes, mucus, feces, nipple secretions, sputum, tears or urine.

27. The method of claim 1, wherein two to nine markers are detected.

28. A method of diagnosing prostate cancer or benign prostate hyperplasia, comprising:
(a) detecting in a test sample from a subject protein biomarkers in the following groups and having the following molecular weights:
(i) about 7024±13 Dalton and about 7820±14 Dalton;
(ii) about 7820±14 Dalton, about 7024±13 Dalton, about 5382±97 Dalton and about 4475±81 Dalton;
(iii) about 8141±15 Dalton, about 9149±16 Daltons, and about 9656±17 Dalton;
(iv) about 9149±16 Dalton and about 9508±17 Dalton;
(v) about 5074±91 Dalton, about 9149±16 Dalton and about 9656±17 Dalton; or
(vi) about 5382±97 Dalton, about 7024±13 Dalton and about 7820±14 Dalton; and
(b) correlating the determination to a diagnosis of benign prostate hyperplasia, prostate cancer or a negative diagnosis.

29. The method of claim 28, wherein said detecting is performed by mass spectroscopy.

30. The method of claim 28, wherein said mass spectroscopy is laser desorption mass spectroscopy.

31. The method of claim 29, wherein said mass spectroscopy is surface enhanced laser desorption/ionization mass spectroscopy.

32. The method of claim 30, wherein the laser desorption/ionization mass spectroscopy includes:
(a) providing a substrate comprising an adsorbent attached thereto;
(b) contacting the test sample with the adsorbent;
(c) desorbing and ionizing the biomarkers from the substrate; and
(d) detecting the desorbed/ionized biomarkers with a mass spectrometer.

33. The method of claim 28, further comprising purifying the test sample prior to contacting the test sample with the adsorbent.

34. The method of claim 28, wherein said detecting is performed by an immunoassay.

35. The method of claim 34, wherein said immunoassay is an enzyme immunoassay.

36. The method of claim 28, wherein said method comprises determining the presence of a protein biomarker having a molecular weight of about 7024±13 and the absence of a protein biomarker having a molecular weight of about 7820±14 Daltons.

37. The method of claim 28, wherein said method comprises determining the absence of the about 7820±14 Dalton and about 7024±13 Dalton biomarkers, the presence of the about 5382±97 Dalton biomarker and the quantity of the about 4475±81 Dalton biomarker.

38. The method of claim 28, wherein said method comprises determining the quantity of the about 8141±15 Dalton, about 9149±16 Dalton, and about 9656±17 Dalton biomarkers.

39. The method of claim 28, wherein said method comprises determining the quantity of the about 9149±16 Dalton and about 9508±17 Dalton biomarkers.

40. The method of claim 28, wherein said method comprises determining the quantity of the about 5074±91 Dalton, about 9149±16 Dalton and about 9656±17 Dalton biomarkers.

41. The method of claim 28, wherein said method comprises determining the absence of the about 5382±97 Dalton, about 7024±13 Dalton and about 7820±14 Dalton biomarkers.

42. The method of claim 28, wherein biomarkers from one to six of said groups are detected.

43. A method for diagnosing benign prostate hyperplasia or prostate cancer, comprising:
(a) detecting at least two protein biomarkers in a test sample from a subject, said protein biomarkers having a molecular weight selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons; and
(b) correlating the detection to a diagnosis of benign prostate hyperplasia, prostate cancer or a negative diagnosis.

44. The method of claim 43, wherein said method comprises:
(a) detecting protein biomarkers having a molecular weight selected from the group consisting of about 3963±7, about 4079±7, about 6542±12, about 6797±12, about 6949±13; about 6990±13, about 7024±13, about 7885±14, about 8067±15, about 8356±15, about 9656±17, about 9720±18 Daltons, and a combination thereof; and
(b) correlating the detection to a diagnosis of prostate cancer.

45. The method of claim 43, wherein said method comprises detecting protein biomarkers having a molecular weight selected from the group consisting of about 3486±6, about 4071±7, about 4580±8, about 5298±10, about 6099±11, about 7054±13, about 7820±14, about 7844±14, about 8943±16, and a combination thereof; and
(b) correlating the detection to a diagnosis of benign prostate hyperplasia.

46. The method of claim 43, wherein said method comprises detecting from two to twelve of said protein biomarkers.

47. The method of claim 45, wherein said method comprises detecting from two to nine of said protein biomarkers.

48. The method of claim 43, wherein said method comprises detecting from two to twenty-one of said protein biomarkers.

49. A kit, comprising:
(a) a substrate comprising an adsorbent attached thereto, wherein the adsorbent is capable of retaining at least one protein biomarker selected from the group consisting of about 4475±81, about 5074±91, about 5382±97, 7024±13, about 7820±14, about 8141±15, about 9149±16, about 9508±17, and about 9656±17 Daltons; and
(b) instructions to detect the protein biomarker by contacting a test sample with the adsorbent and detecting the biomarker retained by the adsorbent.

50. The kit of claim 49, wherein the substrate is a probe adapted for use with a gas phase ion spectrometer, said probe having a surface onto which the adsorbent is attached.

51. The kit of claim 49, wherein the adsorbent is a metal chelate adsorbent.

52. The kit of claim 49, wherein the adsorbent comprises a cationic group.

53. The kit of claim 49, wherein the substrate comprises a plurality of different types of adsorbent.

54. The kit of claim 49, wherein the adsorbent is an antibody that specifically binds to the biomarker.

55. The kit of claim 49, wherein the kit further comprises (1) an eluant wherein the biomarker is retained on the adsorbent when washed with the eluant.

56. A kit, comprising:
(a) a substrate comprising an adsorbent attached thereto, wherein the adsorbent is capable of retaining at least one protein biomarker selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons; and
(b) instructions to detect the protein biomarker by contacting a test sample with the adsorbent and detecting the biomarker retained by the adsorbent.

57. The kit of claim 49, wherein the substrate is a probe adapted for use with a gas phase ion spectrometer, said probe having a surface onto which the adsorbent is attached.

58. The kit of claim 49, wherein the adsorbent is a metal chelate adsorbent.

59. The kit of claim 49, wherein the adsorbent comprises a cationic group.

60. The kit of claim 49, wherein the substrate comprises a plurality of different types of adsorbent.

61. The kit of claim 49, wherein the adsorbent is an antibody that specifically binds to the biomarker.

62. The kit of claim 49, wherein the kit further comprises (1) an eluant wherein the biomarker is retained on the adsorbent when washed with the eluant.

63. A method of using a plurality of classifiers to make a probable diagnosis of prostate cancer, benign prostate hyperplasia, or a negative diagnosis, comprising the steps of:
a) obtaining mass spectra from a plurality of samples from normal subjects, subjects diagnosed with prostate cancer, and subjects diagnosed with benign prostate hyperplasia; and;
b) applying a boosted decision tree analysis to at least a portion of the mass spectra to obtain a plurality of weighted base classifiers comprising a peak intensity value and an associated threshold value; and
c) making a probable diagnosis of at least one of prostate cancer, benign prostate hyperplasia, and a negative diagnosis based on a linear combination of the plurality of weighted base classifiers.

64. A method of developing a plurality of classifiers for use in making a probable diagnosis of prostate cancer, benign prostate hyperplasia, or a negative diagnosis, comprising the steps of:
a) obtaining mass spectra from a plurality of samples from normal subjects, subjects diagnosed with prostate cancer, and subjects diagnosed with benign prostate hyperplasia; and
b) applying a boosted decision tree analysis to at least a portion of the mass spectra to obtain a plurality of weighted base classifiers comprising a peak intensity value and associated threshold value, said values used in linear combination to make a probable diagnosis of at least one of prostate cancer and a negative diagnosis.

65. A computer program medium storing computer instructions therein for instructing a computer to perform a computer-implemented process using a plurality of classifiers to make a probable diagnosis of prostate cancer, benign prostate hyperplasia, or a negative diagnosis, comprising:
a) first computer program code means for obtaining mass spectra from a plurality of samples from normal subjects, subjects diagnosed with prostate cancer, and subjects diagnosed with benign prostate hyperplasia;
b) second computer program code means for applying a boosted decision tree analysis to at least a portion of the mass spectra to obtain a plurality of weighted base classifiers comprising a peak intensity value and an associated threshold value; and
c) third computer program code means for making a probable diagnosis of at least one of prostate cancer, benign prostate hyperplasia, and a negative diagnosis based on a linear combination of the plurality of weighted base classifiers.

66. A computer program medium storing computer instructions therein for instructing a computer to perform a computer-implemented process for developing a plurality of classifiers for use in making a probable diagnosis of prostate cancer, benign prostate hyperplasia, or a negative diagnosis, comprising:
a) first computer program code means for obtaining mass spectra from a plurality of samples from normal subjects, subjects diagnosed with prostate cancer, and subjects diagnosed with benign prostate hyperplasia; and
b) second computer program code means for applying a boosted decision tree analysis to at least a portion of the mass spectra to obtain a plurality of weighted base classifiers comprising a peak intensity value and associated threshold value, said values used in linear combination to make a probable diagnosis of at least one of prostate cancer and a negative diagnosis.

67. A computer program medium storing computer instructions therein for instructing a computer to perform a computer-implemented process of aiding in a diagnosis of benign prostate hyperplasia or prostate cancer, comprising:
(a) first computer program code means for detecting at least two protein biomarkers in a test sample from a subject, said protein biomarkers having a molecular weight selected from the group consisting of about 4475±81, about 5074±91, about 5382±97, 7024±13, about 7820±14, about 8141±15, about 9149±16, about 9508±17, and about 9656±17 Daltons; and
(b) second computer program code means for correlating the detection with a probable diagnosis of benign prostate hyperplasia, prostate cancer or a negative diagnosis.

68. The medium of claim 67, wherein at least two protein biomarkers are the about 7820±14 Dalton and the 7024±13 Dalton protein biomarkers.

69. The medium of claim 67, wherein the at least two protein biomarkers are the about 7820±14 Dalton, the about 7024±13 Dalton, the about 5382±97 Dalton, and the about 4475±81 Dalton biomarkers.

70. The medium of claim 67, wherein the at least two protein biomarkers are the about 8141±15 Dalton, about 9149±16 Dalton, and the about 9656±17 Dalton biomarkers.

71. The medium of claim 67, wherein the at least two protein biomarkers are the about 5074±91 Dalton, the about 9149±16 Dalton, and the about 9656±17 Dalton biomarkers.

72. The medium of claim 67, wherein the at least two protein biomarkers are the about 9149±16 Dalton and the about 9508±17 Dalton biomarkers.

73. The medium of claim 67, wherein the at least two protein biomarkers are the about 5382±97 Dalton, the about 7024±13 Dalton, and the about 7820±14 Dalton biomarkers.

74. The medium of claim 67, wherein the at least two protein biomarkers are the about 7024±13 Dalton, the about 7820±14 Dalton, the about 5382±97 Dalton, and the about 4475±81 Dalton biomarkers.

75. The medium of claim 68, further comprising third computer program code means for determining the absence of the about 7820±14 Dalton protein biomarker and determining the presence of the about 7024±13 Dalton protein biomarker.

76. The medium of claim 69, wherein said medium comprises fourth computer program code means for determining the absence of the about 7820±14 Dalton and about 7024±13 Dalton biomarkers, the presence of the about 5382±97 Dalton biomarker, and the quantity of the about 4475±81 Dalton biomarker.

77. The medium of claim 70, wherein said medium comprises fifth computer program code means for determining the quantity of the about 8141±15 Dalton, about 9149±16 Dalton, and about 9656±17 Dalton biomarkers.

78. The medium of claim 71, further comprising sixth computer program code means for determining the quantity of the about 5074±91 Dalton, about 9149±16 Dalton, and about 9656±17 Dalton biomarkers.

79. The medium of claim 72, further comprising seventh computer program code means for determining the quantity of the about 9149±16 Dalton and about 9508±17 Dalton biomarkers.

80. The medium of claim 73, further comprising eighth computer program code means for determining the absence of the about 5382±97 Dalton, about 7024±13 Dalton and about 7820±14 Dalton biomarkers.

81. The medium of claim 74, further comprising ninth computer program code means for determining the absence of the about 7024±13 Dalton and about 7820±14 Dalton biomarkers, the presence of the about 5382±97 Dalton biomarker and determining the quantity of the about 4475±81 Dalton biomarker.

82. A computer program medium storing computer instructions therein for instructing a computer to perform a computer-implemented process for diagnosing prostate cancer or benign prostate hyperplasia, comprising:
a) first computer program code means for detecting in a test sample from a subject protein biomarkers in the following groups and having the following molecular weights:
(i) about 7024±13 Dalton and about 7820±14 Dalton;
(ii) about 7820±14 Dalton, about 7024±13 Dalton, about 5382±97 Dalton and about 4475±81 Dalton;
(iii) about 8141±15 Dalton, about 9149±16 Daltons, and about 9656±17 Dalton;
(iv) about 9149±16 Dalton and about 9508±17 Dalton;
(v) about 5074±91 Dalton, about 9149±16 Dalton and about 9508±17 Dalton; or
(vi) about 5382±97 Dalton, about 7024±13 Dalton and about 7820±14 Dalton; and
(b) second computer program code means for correlating the determination to a diagnosis of benign prostate hyperplasia, prostate cancer or a negative diagnosis.

83. The medium of claim 82, further comprising third computer program code means for determining the presence of a protein biomarker having a molecular weight of about 7024±13 and the absence of a protein biomarker having a molecular weight of about 7820±14 Daltons.

84. The medium of claim 82, further comprising fourth computer program code means for determining the absence of the about 7820±14 Dalton and about 7024±13 Dalton biomarkers, the presence of the about 5382±97 Dalton biomarker and the quantity of the about 4475±81 Dalton biomarker.

85. The medium of claim 82, further comprising fifth computer program code means for determining the quantity of the about 8141±15 Dalton, about 9149±16 Dalton, and about 9656±17 Dalton biomarkers.

86. The medium of claim 82, further comprising sixth computer program code means for determining the quantity of the about 9149±16 Dalton and about 9508±17 Dalton biomarkers.

87. The medium of claim 82, further comprising seventh computer program code means for determining the quantity of the about 5074±91 Dalton, about 9149±16 Dalton and about 9508±17 Dalton biomarkers.

88. The medium of claim 82, further comprising eighth computer program code means for determining the absence of the about 5382±97 Dalton, about 7024±13 Dalton and about 7820±14 Dalton biomarkers.

89. A computer program medium storing computer instructions therein for instructing a computer to perform a computer-implemented process for diagnosing benign prostate hyperplasia or prostate cancer, comprising:
(a) first computer program code means for detecting at least two protein biomarkers in a test sample from a subject, said protein biomarkers having a molecular weight selected from the group consisting of about 3486±6, about 3963±7, about 4071±7, 4079±7, about 4580±8, about 5298±10, about 6099±11, about 6542±12, about 6797±12, about 6949±13; about 6990±13, about 7024±13, about 7054±13, about 7820±14, about 7844±14, about 7885±14, about 8067±15, about 8356±15, about 8943±16, about 9656±17, and about 9720±18 Daltons; and
(b) second computer program means for correlating the detection to a diagnosis of benign prostate hyperplasia, prostate cancer or a negative diagnosis.

90. The medium of claim 89, further comprising:
(a) third computer program code means for detecting protein biomarkers having a molecular weight selected from the group consisting of about 3963±7, about 4079±7, about 6542±12, about 6797±12, about 6949±13; about 6990±13, about 7024±13, about 7885±14, about 8067±15, about 8356±15, about 9656±17, about 9720±18 Daltons, and a combination thereof; and
(b) fourth computer program code means for correlating the detection to a diagnosis of prostate cancer.

91. The medium of claim 90, further comprising:
(a) third computer program means for detecting protein biomarkers having a molecular weight selected from the group consisting of about 3486±6, about 4071±7, about 4580±8, about 5298±10, about 6099±11, about 7054±13, about 7820±14, about 7844±14, about 8943±16, and a combination thereof; and
(b) fourth computer program code means for correlating the detection to a diagnosis of benign prostate hyperplasia.
